(19) United States
(12) Patent Application Publication
(10) Pub. No.: US 2023/0241123 A1
(43) Pub. Date: Aug. 3, 2023

(54) PLURIPOTENT STEM CELL AND DERIVATIVE THEREOF

(71) Applicants: Linli Wang, Guangzhou, Guangdong (CN); Future Homo Sapiens Institute of Regenerative Medicine Co., Ltd., Guangzhou, Guangdong (CN)

(72) Inventors: Linli Wang, Guangzhou, Guangdong (CN); Yuehua Chen, Guangzhou, Guangdong (CN); Jian Mo, Guangzhou, Guangdong (CN); Jianguo Yang, Guangzhou, Guangdong (CN)

(21) Appl. No.: 17/916,395

(22) PCT Filed: Aug. 13, 2021
(86) PCT No.: PCT/CN2021/112449

§ 371 Date: Apr. 10, 2023

(30) Foreign Application Priority Data
Dec. 4, 2020 (CN) 202011397673.7

(51) Int. Cl. A61K 35/545; C12N 15/113; C12N 15/85; A61K 48/00; A61K 35/28; A61K 35/30

(52) U.S. Cl. CPC A61K 35/545; C12N 15/113; C12N 15/85; A61K 48/0008; A61K 35/28; A61K 35/30; C12N 2310/14; C12N 2310/141

(57) ABSTRACT

The present invention provides a pluripotent stem cell or a derivative thereof. A first nucleic acid molecule is introduced into a genome of the pluripotent stem cell or the derivative thereof; and a second nucleic acid molecule is introduced to a 3′UTR region of an immune response-related gene in the pluripotent stem cell or the derivative thereof. The first nucleic acid molecule encodes a small nucleic acid molecule that mediates RNA interference, and the small nucleic acid molecule can specifically bind to a transcript product of the second nucleic acid molecule to start an RNA interference program to degrade or silence mRNA of the immune response-related gene, thereby blocking the expression of the immune response-related gene, such that the cell has immunological compatibility, and thus can eliminate or reduce alloimmune rejection responses.

### CROSS-REFERENCE TO RELATED APPLICATIONS

The present application is a national phase entry under 35 USC § 371 of International Application PCT/CN2021/112449, filed on Aug. 13, 2021, which claims the benefit of and priority to Chinese Patent Application No. 202011397673.7, filed on Dec. 4, 2020, the entire disclosures of which are incorporated herein by reference.

### INCORPORATION BY REFERENCE

This application includes a sequence listing in computer readable form (a “txt” file) that is submitted herewith on ASCII text file named Sequence Listing .txt, created on Sep. 28, 2022 and 12,627 bytes in size. This sequence listing is incorporated by reference herein.

### TECHNICAL FIELD

The present disclosure belongs to the technical field of genetic engineering, and relates to an immunologically compatible pluripotent stem cell or a derivative thereof.

### BACKGROUND

Stem cells are a class of “seed” cells that have the ability to self-renew and differentiate into specific functional somatic cells, have the potential to regenerate into various tissues and organs and the human body, and play a central and irreplaceable role in major biological activities such as immune response, aging, and tumorigenesis. According to the degree of stem cell characteristics, stem cells are mainly classified into totipotent stem cells, pluripotent stem cells (PSCs) and adult stem cells.

Among them, pluripotent stem cells (PSCs) have almost unlimited self-renewal capacity and the potential to develop and differentiate into organs, tissues and cells of all germ layers in an embryo under normal developmental conditions. Typical PSCs mainly include embryonic stem cells (ESCs), embryonic germ cells (EGCs), embryonic carcinoma cells (ECCs), and induced pluripotent stem cells (iPSCs), etc., and such cells have very profound and broad application prospects due to their powerful functions and can pass ethical restrictions to a certain extent.

Due to the huge application prospects of PSCs, industry-university-research work such as PSC bank construction is increasingly developing. However, the conception or establishment of either autologous iPSC cell banks or immunomatched PSC cell banks requires enormous financial, material and human resources. The molecular and immunological basis of transplantation of organ, tissue or cell from allogeneic donors to recipients is mainly based on the matching of the classical major histocompatibility complexes MHC-I and MHC-II (also known as HLA-I and HLA-II in humans). As of June 2019, more than 20,000 alleles of the HLA system have been identified and named, wherein the numbers of only the classical HLA-A, B, and C alleles have respectively exceeded 5,000. The number of possible random combinations of these classical HLA-I/II alleles will be astronomical. With the discovery of new alleles, the number of combinations will increase, which brings great obstacles to tissue matching and donor selection before transplantation, and also brings huge difficulties to the construction of population-covering immunomatched PSC cell banks.

Therefore, the construction of allogeneic immunologically compatible universal PSCs is imminent. In recent years, many reports have indicated that by knocking out genes such as B2M and CIITA, the expression of HLA-I and HLA-II on the cell surface or the genes themselves becomes absent, which in turn enables the cells to develop immune tolerance or escape T/B cell-specific immune responses and generate immunologically compatible universal PSCs, laying an important foundation for the wider application of universal PSC-derived cells, tissues, and organs. It has also been reported that cells overexpress CTLA4-Ig and PD-L1 to inhibit allogeneic immune rejection. Recently, it has also been reported that by knocking in CD47 while knocking out B2M and CIITA, cells develop immune tolerance or escape from innate immune responses by NK cells, etc., in addition to obtaining escape from specific immune responses, such that the cells have more comprehensive and stronger immunological compatibility properties. However, these solutions either have incomplete immunological compatibility and still have allogeneic immune rejections through other routes; or completely eliminate allogeneic immune rejection responses, but at the same time cause the cells of the donor-derived transplant to lose the antigen-presenting ability, which poses a great risk of diseases such as tumorigenicity and viral infection to the recipient.

In this regard, it has also been reported that, instead of directly knocking out B2M, HLA-A and HLA-B(or together with CIITA) are knocked out, while retaining HLA-C, and construct 12 HLA-C immunomatched antigens which cover more than 90% of the human population, such that the transplanted cells retain a certain degree of antigen-presenting function and at the same time can inhibit the innate immune response of NK cells through HLA-C. However, with regard to such cells, first of all, antigen types presented by HLA-I are reduced by more than two-thirds, the integrity of antigens that can be presented is greatly and irreversibly reduced, thereby causing highly biased presentation of antigens in various tumors, viruses and other diseases and retaining a considerable degree of risk of diseases such as tumorigenesis and viral infections remains, which results in that the risk of diseases is even higher when CIITA is also knocked out; secondly, the 12 high-frequency immunomatched HLA-C antigens are highly ethnically diverse, according to our verification and calculation, the proportion can be only 70% in some regions, in addition, there has been no authoritative HLA data display from large sample sizes in China, India and other populous countries, so there is still a huge matching vacancy challenge for using the universal PSCs which are prepared; and thirdly, this method involves repeated gene-editing efforts many times. Based on at least two rounds of single-cell isolation and culture for each gene editing, the entire process requires six or more rounds of single-cell isolation and culture. Due to multiple off-target gene editing, or chromatin instability, or passage and proliferation of large numbers of single cells, these processes inevitably and with great probability cause various unpredictable mutations in cells, and lead to various problems such as carcinogenesis and metabolic diseases. Thus, such immunological compatibility solutions are also expedient measures in the “transition period”, and there are still many problems that have not been better resolved.

In addition, some people have designed suicide genes to induce killing after donor tissue and cells become diseased. The consequences thereof are severe tissue necrosis, cytokine storms and other unpredictable disease risk problems, and another big challenge is that after such designed cells are killed, no more suitable donor cells, tissues and organs will exist.

RNA interference is a ubiquitous natural phenomenon in eukaryotes, in which double-stranded RNA (dsRNA) is used in organisms to induce specific degradation of mRNA of homologous target genes, resulting in gene silencing. During the process of RNAi, long-chain dsRNA is cleaved by the Dicer enzyme into small interfering RNA (siRNA), one strand of the siRNA binds to RNA-induced silencing complex (RISC) and subsequently binds to target mRNA in a complementary base-pairing fashion, resulting in the degradation of the mRNA. Since siRNA can inhibit the expression of specific genes, this technology has been widely used in various research fields, including gene therapy research.

Currently, small nucleic acid molecules known to mediate RNA interference include short interfering nucleic acid (siNA), short interfering RNA (siRNA), and double-stranded RNA (dsRNA).

### SUMMARY

The first object of the present disclosure is to provide an immunologically compatible pluripotent stem cell or a derivative thereof.

The second object of the present disclosure is to provide an immunologically compatible and reversible pluripotent stem cell or a derivative thereof.

The third object of the present disclosure is to provide a use of the above-mentioned pluripotent stem cell or the derivative thereof in the preparation of a product for cellular therapy.

The fourth object of the present disclosure is to provide a use of the above-mentioned pluripotent stem cell or the derivative thereof in the preparation of a product for organ transplantation.

The fifth object of the present disclosure is to provide a use of the above-mentioned pluripotent stem cell or the derivative thereof in the construction of a universal PSC cell bank.

The sixth object of the present disclosure is to provide a use of the above-mentioned pluripotent stem cell or the derivative thereof as a gene drug carrier.

Technical Solution adopted by the disclosure:

The first aspect of the present disclosure provides a pluripotent stem cell or a derivative thereof, wherein a first nucleic acid molecule is introduced into the genome of the pluripotent stem cell or the derivative thereof;

and a second nucleic acid molecule is introduced into a 3′UTR region of an immune response-related gene in the pluripotent stem cell or the derivative thereof,

wherein the first nucleic acid molecule encodes a small nucleic acid molecule that mediates RNA interference, the small nucleic acid molecule specifically targets a transcript of the second nucleic acid molecule, and the small nucleic acid molecule does not target any other mRNA or lncRNA of the pluripotent stem cell or the derivative thereof.

In the present technical solution, the small nucleic acid molecule encoded by the first nucleic acid molecule can specifically bind to the transcript of the second nucleic acid molecule introduced into the 3′UTR region of the immune response-related gene, thereby initiating an RNA interference program to degrade or silence mRNA of the immune response-related gene, and blocking the expression of the immune response-related gene, such that the cells have immunological compatibility and the allogeneic immune rejection response can be eliminated or reduced. Furthermore, the RNA interference program acts only on such engineered pluripotent stem cells or derivatives thereof. Therefore, when such cells or derivatives are transplanted into a recipient, the RNA interference against the immune response-related gene only acts on the donor cells, and dose not interfere with the genome of the cells of the recipient; wherein the RNA interference against the immune response-related gene is mediated by the small nucleic acid molecule (encoded by the first nucleic acid molecule) and the second nucleic acid molecule (introduced into the 3′UTR of the immune response-related gene).

The second aspect of the present disclosure provides a pluripotent stem cell or a derivative thereof, wherein an inducible gene expression system and a first nucleic acid molecule are introduced into the genome of the pluripotent stem cell or the derivative thereof;

and a second nucleic acid molecule is introduced into a 3′UTR region of an immune response-related gene in the pluripotent stem cell or the derivative thereof, wherein the first nucleic acid molecule encodes a small nucleic acid molecule that mediates RNA interference, the small nucleic acid molecule specifically targets a transcript of the second nucleic acid molecule, and the small nucleic acid molecule does not target any other mRNA or lncRNA of the pluripotent stem cell or the derivative thereof and the inducible gene expression system regulates the expression of the first nucleic acid molecule.

In the above technical solution, the inducible gene expression system is regulated by an exogenous inducer; and the on and off state of the inducible gene expression system is controlled by adjusting the added amount, duration of action, and type of the exogenous inducer, so as to control the expression amount of the small nucleic acid molecule.

When the inducible gene expression system is turned on, the normally expressed small nucleic acid molecule specifically binds to the transcript of the second nucleic acid molecule introduced into the 3′UTR region of the immune response-related gene, thereby initiating an RNA interference program to degrade or silence mRNA of the immune response-related gene, and blocking the expression of the immune response-related gene. Therefore, when such cells or derivatives are transplanted into a recipient, the allogeneic immune rejection response can be eliminated or reduced, thereby improving the immunological compatibility between the transplant and the recipient.

When the transplant becomes diseased, the inducible gene expression system can be turned off by adding an exogenous inducer, and the expression of the small nucleic acid molecule is turned off to stop the interference effect of the small nucleic acid molecule on the mRNA of the immune response-related gene, thereby restoring the normal expression of the immune response-related gene, causing the antigen-presenting ability of the transplant cells to be restored, which enables the recipient to eliminate the diseased transplant, thus improving the clinical safety of such pluripotent stem cells or derivatives thereof and greatly expanding the value thereof in clinical applications.

Furthermore, the RNA interference program acts only on such engineered pluripotent stem cells or derivatives thereof; therefore, when such cells or derivatives are transplanted into a recipient, the RNA interference against the immune response-related gene, as mediated by the small nucleic acid molecule encoded by the first nucleic acid molecule and the second nucleic acid molecule introduced into the 3′UTR of the immune response-related gene, only acts on the donor cells, without interfering with the genome of the cells of the recipient.

The inducible gene expression system can be at least one selected from the group consisting of Tet-Off system and dimer-induced expression system.

Where the inducible gene expression system used is the Tet-Off system, the expression of the small nucleic acid molecule in the cell or the derivative thereof can be controlled by adding the exogenous inducer doxycycline (Dox). After the pluripotent stem cells or the derivatives thereof are transplanted into a recipient, it is even possible to adjust the added amount of Dox to gradually reduce the expression amount of the small nucleic acid molecule, such that the cells can progressively express low concentrations of the immune-related gene to stimulate the recipient, thereby enabling the recipient to gradually develop tolerance to the transplanted cells or the derivative thereof and finally achieving a stable tolerance. Typically, Dox is added in an amount of 0-100 uM.

Where the inducible gene expression system used is the dimer-induced expression system, the expression of the small nucleic acid molecule in the cell or the derivative thereof can be controlled by adding the exogenous inducer rapamycin (or an analog thereof). Similarly, it is possible to adjust the added amount of rapamycin (or an analog thereof) to gradually reduce the expression amount of the small nucleic acid molecule, such that the cells can correspondingly express low concentrations of the immune-related gene to stimulate the recipient, whereby the recipient gradually develops tolerance to the transplanted cells or the derivative thereof and finally achieves a stable tolerance. Typically, rapamycin (or an analog thereof) is added in an amount of 0-1000 nM.

Regarding the first and second aspects of the present disclosure, an CD47 expression sequence is further introduced into the genome of the pluripotent stem cell or the derivative thereof, such that the pluripotent stem cell or the derivative thereof can overexpress CD47.

CD47 is an extracellular ligand of human inhibitory receptor signal regulatory protein (SIRP). CD47 binds with its specific ligand SIRPα to form a CD7-SIRPα signal complex, which can send an anti-phagocytic signal to inhibit phagocytosis by phagocytes. CD47 can suppress the activity of macrophage and NK cells, creating a loophole in the immune system and triggering negative regulatory signals. CD47 is a co-stimulator of T cell activation, which activates the T cell apoptosis process, induces T cell anergy, and enhances the efficiency of TCR signaling. CD47 is a very potent non-HLA ligand that can silence all innate immune responses. Overexpression of CD47 in the pluripotent stem cell or the derivative thereof can further improve the immunological compatibility or immune tolerance of the pluripotent stem cell or the derivative thereof.

Regarding the first and second aspects of the present disclosure, the immune response-related gene includes:

(1) major histocompatibility complex-related genes, including at least one selected from the group consisting of B2M and CIITA, wherein:

B2M: Taking β2 microglobulin as an example, the gene named B2M (beta-2-microglobulin) constitutes a light chain part (β chain) of HLA class I molecules. β2-m promotes the transport of the entire HLA molecule from the endoplasmic reticulum to the surface of the cell membrane and can also maintain the structural stability of the entire HLA molecule during the expression of the HLA class I molecule on the cell surface, and β2-m is a subunit necessary for its surface expression, assembly and stability. Many studies have reported that after the B2M gene is knocked out from a cell, HLA class I molecules are no longer expressed on the cell surface, and allogeneic HLA mismatching immune responses associated with the HLA class I molecules disappear accordingly. For HLA I-unmatched cell, tissue or organ transplants (referred to as “transplants”), the recipient will develop immunological compatibility/tolerance to the unmatched HLA I, thus achieving the construction of immunologically compatible pluripotent stem cells or derivatives thereof. However, after complete knockout of B2M, the cell completely loses the ability to present antigens via HLA class I molecules, and there is no way to deal with various lesions (such as canceration) in cells, tissues or organs that may occur after transplantation. This greatly affects the application safety and is not conducive to further clinical applications.

CIITA: CIITA, the gene of class II major histocompatibility complex transactivator, is a key gene known for class II HLA gene transcription, is constitutively expressed in antigen-presenting cells, and may also be expressed in other cells under IFN-γ induction. Cells mainly regulate the expression level of HLA class II genes by regulating the expression of CIITA and thus regulate the intensity of immune response. As a co-activating molecule, CIITA is recruited to the vicinity of a promoter for HLA class II gene and participates in the regulation of the expression and transcription of the gene. The expression level of CIITA is positively correlated with the expression level of HLA II, and thus, it is considered to be the most critical regulatory molecule for HLA class II gene expression.

(2) major histocompatibility complex genes, including at least one selected from the group consisting of HLA-A, HLA-B, HLA-C, HLA-DRA, HLA-DRB1, HLA-DRB3, HLA-DRB4, HLA-DRB5, HLA-DQA1, HLA-DQB1, HLA-DPA1 and HLA-DPB1;

Class I molecules (A, B, and C) are distributed on the surface of all nucleated cells, while class II molecules (DR, DQ, and DP) are only expressed on the surface of some specific cells in lymphoid tissues, such as professional antigen-presenting cells (including B cells, macrophages, and dendritic cells), thymic epithelial cells, and activated T cells. Major histocompatibility antigens (MHC antigens) are transplantation antigens that can cause a strong rejection response, namely HLA molecules encoded by the HLA complex (human MHC). Actually, the difference in HLA type between the donor and the recipient is the main cause of acute transplant rejection. Almost all the classical HLAs are associated with transplant rejection, and especially, class I molecules are extremely important.

Regarding the first and second aspects of the present disclosure, the small nucleic acid molecule includes short interfering nucleic acid (siNA), short interfering RNA (siRNA), and double-stranded RNA (dsRNA), preferably at least one selected from the group consisting of miRNA, shRNA, and shRNA-miR. Among them:

Short interfering nucleic acid (siNA): siNA is a general term for all small nucleic acid molecules with RNA interference (RNAi) effects, and typically refers to a small ribonucleic acid consisting of 20 or more nucleotides.

Short interfering RNA (siRNA): Small interfering RNA (siRNA), sometimes called short interfering RNA or silencing RNA, is a double-stranded RNA of 20 to 25 nucleotides in length, which has many different uses in biology. It is currently known that siRNA is mainly involved in the phenomenon of RNA interference (RNAi) and regulates gene expression in a specific manner.

Double-stranded RNA (dsRNA): dsRNA is an RNA having a complementary strand and is similar to DNA found in cells, and dsRNA makes up the genome of some viruses (double-stranded RNA viruses). Double-stranded RNA like viral RNA or siRNA can trigger RNA interference in eukaryotic cells and elicit interferon responses in vertebrates.

miRNA: miRNA is a single-stranded RNA fragment between 21-23 nucleotides in length that regulates gene expression. A miRNA is encoded by a gene and is transcribed from DNA but not translated into a protein. Primary transcripts (pri-miRNAs) are cut down from their cervical-loop structures to obtain functional miRNAs. Mature miRNA molecules are partially complementary to one or more mRNA molecules, and the main function thereof is to down-regulate gene expression.

shRNA: shRNA consists of two short reverse complementary sequences, separated by a stem-loop (loop) sequence in the middle, thereby forming a hairpin structure; shRNA is controlled by pol III promoter, then connected to 5-6 Ts as a transcription terminator of RNA polymerase III, and it is often used for RNA interference to silence the expression of target genes.

shRNA-miR: Based on the design of the double-arm structure of miR-30 or miR-155 and the stem-loop structure of shRNA, the target sequence of microRNA is replaced with the target sequence of shRNA.

When the pluripotent stem cell or derivative thereof is derived from human, the sequence of the small nucleic acid molecule is accordingly a random sequence derived from a non-human species that does not target any human mRNA or lncRNA, preferably from Caenorhabditis elegans. For example,

(SEQ ID NO. 1)
5′-TTGTACTACACAAAAGTACTG-3′
(SEQ ID NO. 3)
5′-TCACAACCTCCTAGAAAGAGTAGA-3′

The first nucleic acid molecule and the second nucleic acid molecule designed according to the above sequences are any of the following combinations:

### Combination I:

Small nucleic acid molecule sequence: 5′-TTGTACTACACAAAAGTACTG-3′ (SEQ ID NO. 1)

(1) First nucleic acid molecule (i.e., the shRNA expression framework or the shRNA-miR expression framework of the small nucleic acid molecule):

shRNA expression framework: It comprises two reverse complementary small nucleic acid molecule sequences, separated by a stem-loop sequence in the middle, thereby forming a hairpin structure, followed by 5-6 Ts as an transcription terminator of RNA polymerase III.

shRNA-miR expression framework: The original target sequence of miR-30 or miR-155 is replaced with the sequence of the small nucleic acid molecule.

(2) Second nucleic acid molecule: It includes at least 3 repeats of the reverse complement sequence of the small nucleic acid molecule sequence, preferably 6-10 repeats of the reverse complement sequence of the small nucleic acid molecule sequence. The reverse complement sequence of the small nucleic acid molecule sequence can be linked by a random linker sequence.

As an embodiment of the present disclosure, the second nucleic acid molecule is formed of a 10 nt random sequence and 8 repeats of the reverse complementary sequence of the small nucleic acid molecule sequence linked via random linker sequences (CGTA):

(SEQ ID NO. 2)
atTCTAGATACAGTACTTTTGTGTAGTACAACGTACAGTACTTTTGTGT
AGTACAACGTACAGTACTTTTGTGTAGTACAACGTACAGTACTTTTGTG
TAGTACAACGTACAGTACTTTTGTGTAGTACAACGTACAGTACTTTTGT
GTAGTACAACGTACAGTACTTTTGTGTAGTACAACGTACAGTACTTTTG
TGTAGTACAACGTA

### Combination II:

Small nucleic acid molecule sequence: 5′-TCACAACCTCCTAGAAAGAGTAGA-3′ (SEQ ID NO. 3)

(1) First Nucleic Acid Molecule:

shRNA expression framework: It comprises two reverse complementary small nucleic acid molecule sequences, separated by a stem-loop sequence in the middle, thereby forming a hairpin structure, followed by 5-6 Ts as an transcription terminator of RNA polymerase.

shRNA-miR expression framework: The original target sequence of microRNA mir-30 or mir-155 is replaced with the sequence of the small nucleic acid molecule.

(2) Second nucleic acid molecule: It includes at least three repeats of the reverse complement sequence of the small nucleic acid molecule sequence, preferably 6-10 repeats of the reverse complement sequence of the small nucleic acid molecule sequence. The reverse complement sequence of the small nucleic acid molecule sequence can be linked by a random linker sequence.

As an embodiment of the present disclosure, the second nucleic acid molecule is formed of a 10 nt random sequence and 8 repeats of the reverse complementary sequence of the small nucleic acid molecule sequence linked via random linker sequences (CGTA):

(SEQ ID NO. 4)
atTCTAGATATCTACTCTTTCTAGGAGGTTGTGACGTATCTACTCTTTC
TAGGAGGTTGTGACGTATCTACTCTTTCTAGGAGGTTGTGACGTATCTA
CTCTTTCTAGGAGGTTGTGACGTATCTACTCTTTCTAGGAGGTTGTGAC
GTATCTACTCTTTCTAGGAGGTTGTGACGTATCTACTCTTTCTAGGAGG
TTGTGACGTATCTACTCTTTCTAGGAGGTTGTGACGTA

Regarding the first and second aspects of the present disclosure, the introduction loci of the first nucleic acid molecule and/or the inducible gene expression system are genomic safe loci.

Preferably, the genomic safe loci include at least one selected from the group consisting of the AAVS1 safe locus, the eGSH safe locus, and the H11 safe locus. Among them:

The AAVS1 locus (alias “PPP1R2C locus”), located on chromosome 19 of the human genome, is a validated “safe harbor” locus that could ensure the intended function of the transferred DNA fragment. This locus is an open chromosomal structure, which could ensure that the transferred gene could be transcribed normally, and the insertion of an exogenous fragment of interest at this locus has no known side effects on cells.

The eGSH safe locus, located on chromosome 1 of the human genome, is another documented validated “safe harbor” locus that could ensure the intended function of the transferred DNA fragment.

The H11 safe locus (also called Hipp11), located on human chromosome 22, is a locus between the two genes Eif4enif1 and Drg1. The locus was discovered and named by Simon Hippenmeyer in 2010. Since the H11 locus is located between the two genes, the risk of affecting the expression of endogenous genes after the insertion of an exogenous gene is small. The H11 locus is verified to be an intergenic safe transcription activation region, and a new “safe harbor” locus other than the AAVS1 locus and the eGSH locus.

Regarding the first and second aspects of the present disclosure, the first nucleic acid molecule and/or the inducible gene expression system are introduced by means of viral vector interference, non-viral vector transfection or gene editing.

Regarding the first and second aspects of the present disclosure, the second nucleic acid molecule is introduced by means of gene editing.

The gene editing is preferably gene knock-in.

The third aspect of the present disclosure provides a use of the pluripotent stem cell or the derivative thereof of the first or second aspect in the preparation of a product for cellular therapy.

The fourth aspect of the present disclosure provides a use of the pluripotent stem cell or the derivative thereof of the first or second aspect in the preparation of a product for organ transplantation.

The fifth aspect of the present disclosure provides a use of the pluripotent stem cell or the derivative thereof of the first or second aspect in the construction of a universal PSC cell bank.

The sixth aspect of the present disclosure provides a use of the pluripotent stem cell or the derivative thereof of the first or second aspect as a gene drug carrier.

### Beneficial Effects of the Disclosure

The pluripotent stem cells or the derivatives thereof provided in the first aspect of the present disclosure have immunological compatibility properties and can eliminate or reduce the allogeneic immune rejection response. Moreover, the RNA interference program of the pluripotent stem cells or derivatives thereof only acts on such engineered pluripotent stem cells or derivatives thereof. Therefore, when such cells or derivatives are transplanted into a recipient, the RNA interference against the immune response-related gene, as mediated by the small nucleic acid molecule encoded by the first nucleic acid molecule and the transcript of the second nucleic acid molecule, only acts on the donor cells, without interfering with the genome of the cells of the recipient.

The pluripotent stem cells or derivatives thereof provided by the second aspect of the present disclosure have the characteristics of immunological compatibility reversibility. When the inducible gene expression system is turned on, the normally expressed small molecule nucleic acid specifically binds to the transcript of the second nucleic acid molecule introduced into the 3′UTR region of the immune response-related gene, thereby initiating an RNA interference program to degrade or silence mRNA of the immune response-related gene, and blocking the expression of the immune response-related gene. Therefore, when such cells or derivatives are transplanted into a recipient, the allogeneic immune rejection response can be eliminated or reduced, thereby improving the immunological compatibility between the transplant and the recipient.

When the transplant becomes diseased, the inducible gene expression system can be turned off by adding an exogenous inducer, and the expression of the small nucleic acid molecule and the interference effect of the small nucleic acid molecule on the mRNA of the immune-related gene are stopped, thereby restoring the normal expression of the immune-related gene, causing the antigen-presenting ability of the transplant cells to be restored, which enables the recipient to eliminate the diseased transplant, thus improving the clinical safety of such pluripotent stem cells or derivatives thereof and greatly expanding the value thereof in clinical applications.

The immunologically compatible and reversible pluripotent stem cell or derivative thereof of the present disclosure also has another very significant advantage, i.e., it is possible to adjust the added amount and duration of action of the exogenous inducer to gradually reduce the expression amount of the small nucleic acid molecule in the pluripotent stem cell or the derivative thereof, such that the recipient cells can gradually express low concentrations of the immune-related gene to stimulate the recipient, whereby the recipient gradually develops tolerance to the transplanted cells or the derivative thereof and finally achieves a stable tolerance. At this point, even if the transplant cells express mismatched HLA class I molecules on their surface, they can also be compatible with the recipient's immune system.

### BRIEF DESCRIPTION OF DRAWINGS

FIG. 1 shows a Cas9(D10A) plasmid map.

FIG. 2 shows a sgRNA Clone AAVS1-1 plasmid map.

FIG. 3 shows a sgRNA Clone AAVS1-2 plasmid map.

FIG. 4 shows a sgRNA clone B2M-1 plasmid map.

FIG. 5 shows a sgRNA clone B2M-2 plasmid map.

FIG. 6 shows a sgRNA clone CIITA-1 plasmid map.

FIG. 7 shows a sgRNA clone CIITA-2 plasmid map.

FIG. 8 shows an AAVS1 KI Vector(shRNA,constitutive) plasmid map.

FIG. 9 shows an AAVS1 KI Vector(shRNA,inducible) plasmid map.

FIG. 10 shows an AAVS1 KI Vector(shRNA-miR,constitutive) plasmid map.

FIG. 11 shows an AAVS1 KI Vector(shRNA-miR,inducible) plasmid map.

FIG. 12 shows a B2M KI Vector.

FIG. 13 shows a CIITA KI Vector.

### DETAILED DESCRIPTION

In order to understand the technical content of the present disclosure more clearly, the following examples are specifically given for detailed description in conjunction with the accompanying drawings. It should be understood that these examples are only used to describe the present disclosure, rather than limiting the scope of the present disclosure. Experimental methods in which no specific conditions are indicated in the following examples are usually carried out under conventional conditions, for example, the conditions described in Sambrook et al., Molecular Cloning: A Laboratory Manual (New York: Cold Spring Harbor Laboratory Press, 1989), or in accordance with the conditions recommended by the manufacturer. Various common chemical reagents used in the examples are all commercially available products.

1 Experimental Material

1.1 Starting Stem Cells or Derivatives Thereof

Pluripotent stem cells or derivatives thereof were selected from embryonic stem cells (ESCs), induced pluripotent stem cells (iPSCs) and other forms of pluripotent stem cells, such as hPSCs-MSCs, NSCs, and EB cells, Among them:

iPSCs: pE3.1-OG-KS and pE3.1-L-Myc-hmiR302 cluster were electrotransfected into somatic cells by using our established third-generation efficient and safe episomal-iPSC induction system (6F/BM1-4C), and cultured in RM1 for 2 days, in BioCISO-BM1 with 2 μM Parnate for 2 days, in BioCISO-BM1 with 2 μM Parnate, 0.25 mM sodium butyrate, 3 μM CHIR99021 and 0.5 μM PD03254901 for 2 days. In the stem cell medium BioCISO until approximately 17 days, iPSC clones were picked, and the picked iPSC clones were purified, digested, and passaged to obtain stable iPSCs. For the specific construction method, see: Stem Cell Res Ther. 2017 Nov. 2; 8(1): 245.

hPSCs-MSCs: iPSCs were cultured in a stem cell medium (BioCISO, containing 10 μM TGFβ inhibitor SB431542) for 25 days during which digestion and passage were carried out at 80-90 confluence (2 mg/mL Dispase digestion), the cells were passaged 1:3 into a Matrigel-coated culture plate and then into an ESC-MSC medium (knockout DMEM medium, containing 10% KSR, NEAA, double antibody, glutamine, β-mercaptoethanol, 10 ng/mL bFGF and SB-431542), wherein the medium was changed every day, the passage was carried out at 80-90 confluence (1:3 passage), and the cells were cultured continuously for 20 days. For the specific construction method, see: Proc Natl Acad Sci USA. 2015; 112(2): 530-535.

NSCs: iPSCs were cultured in an induction medium (knockout DMEM medium, containing 10% KSR, TGF-β inhibitor, and BMP4 inhibitor) for 14 days, and rosette-shaped nerve cells were picked and cultured in a low-adherence culture plate, wherein the medium used was DMEM/F12 (containing 1% N2, Invitrogen) and Neurobasal medium (containing 2% B27, Invitrogen) at a ratio of 1:1 and further contained 20 ng/ml bFGF and 20 ng/ml EGF. Accutase was used for digestion and passage. For the specific construction method, see: FASEB J. 2014; 28(11): 4642-4656.

EB cells: iPSCs with a confluence of 95% were digested with BioC-PDE1 for 6 min and then scraped into a mass by a mechanical scraping passage method. Cell mass settling occurred, and the settled cell mass was transferred to a low-adherence culture plate and cultured using BioCISO-EB1 for 7 days during which the medium was changed every other day. After 7 days, the cells were transferred to a Matrigel-coated culture plate to continue adherent culture with BioCISO, and after 7 days, embryoid bodies (EBs) with a structure of inner, middle and outer germ layers could be obtained. For the specific construction method, see: Stem Cell Res Ther. 2017 Nov. 2; 8(1): 245.

The pluripotent stem cell derivatives included adult stem cells differentiated from the pluripotent stem cells, and cells or tissues of each germ layer.

1.2 Small Nucleic Acid Molecule and the Corresponding First Nucleic Acid Molecule, and Second Nucleic Acid Molecule

The sequence of the small nucleic acid molecule was: 5′TTGTACTACAC AAAAGTACTG 3′ (SEQ ID NO. 1).

Those skilled in the art would understand that other random sequences of non-human species that do not target any human mRNA or lncRNA can all achieve the objects of the present disclosure, such as SEQ ID NO. 3.

First Nucleic Acid Molecule (i.e., the shRNA Expression Framework or the shRNA-miR Expression Framework of the Small Nucleic Acid Molecule):

(1) shRNA expression framework: The shRNA expression framework comprised, in sequence from 5′ to 3′, a small nucleic acid molecule sequence, a stem-loop sequence, a reverse complement sequence of the small nucleic acid molecule sequence, and Poly T; the two reverse complementary sequences were separated by the stem-loop sequence in the middle to form a hairpin structure, and finally Poly T was connected as an transcription terminator of RNA polymerase III; and

a promoter sequence and matched promoter regulatory elements were added to a front end of the expression framework according to expression requirements.

The specific sequence was:

(SEQ ID NO. 5)
GAGGGCCTATTTCCCATGATTCCTTCATATTTGCATATACGATACAAGG
CTGTTAGAGAGATAATTGGAATTAATTTGACTGTAAACACAAAGATATT
AGTACAAAATACGTGACGTAGAAAGTAATAATTTCTTGGGTAGTTTGCA
GTTTTAAAATTATGTTTTAAAATGGACTATCATATGCTTACCGTAACTT
GAAAGTATTTCGATTTCTTGGCTTTATATATCTTGTGGAAAGGACTTTA
CCACTCCCTATCAGTGATAGAGAAAAGTGAAAGTCGAGTTTACCACTCC
CTATCAGTGATAGAGAAAAGTGAAAGTCGAGTTTACCACTCCCTATCAG
TGATAGAGAAAAGTGAAAGTCGAGTTTACCACTCCCTATCAGTGATAGA
GAAAAGTGAAAGTCGAGTTTACCACTCCCTATCAGTGATAGAGAAAAGT
GAAAGTCGAGTTTACCACTCCCTATCAGTGATAGAGAAAAGTGAAAGTC
GAGTTTACCACTCCCTATCAGTGATAGAGAAAAGTGAAAGTCGAGCTCG
GTACCCGGGTCGAGGTAGGCGTGTACGGTGGGAGGCCTATATAAGCAGA
GCTCGTTTAGTGAACCGTCAGATCGCCTGGAGACGCCATCCACGCTGTT
TTGACCTCCATAGAAGACACCGGGACCGATCCAGCCTGCTAGCGCCACC
(SEQ ID NO. 6)
N₁...N₂₁TTCAAGAGA
N₂₂...N₄₂TTTTTT

wherein:

a. N₁ . . . N₂₁ was the small nucleic acid molecule sequence, and N₂₂ . . . N₄₂ was the reverse complement sequence of the small nucleic acid molecule sequence;

b. if the plasmid was required to express shRNAs of multiple genes, each gene corresponded to an shRNA expression framework, and they were then seamlessly connected;

c. constitutive shRNA plasmids with different resistance genes were only different in resistance gene, and the other sequences were the same;

d. N represented the base A, T, G, or C;

e. SEQ ID NO. 5 was a promoter sequence; and

f. SEQ ID NO. 6 was a stem-loop sequence.

(2) shRNA-miR expression framework: It was obtained by replacing the target sequence in microRNA-30 or microRNA-155 with the small nucleic acid molecule sequence.

The specific sequence was as follows:

(SEQ ID NO. 7)
GAGGCTTCAGTACTTTACAGAATCGTTGCCTGCACATCTTGGAAACACT
TGCTGGGATTACTTCTTCAGGTTAACCCAACAGAAGGCTAAAGAAGGTA
TATTGCTGTTGACAGTGAGCG
(SEQ ID NO. 8)
M₁N₁...N₂₁TAGTGAAGCCACAGATGTA
(SEQ ID NO. 9)
N₂₂...N₄₂M₂TGCCTACTGCCTCGGACTTCAAGGGGCTACTTTAGGAGCA
ATTATCTTGTTTACTAAAACTGAATACCTTGCTATCTCTTTGATACATT
TTTACAAAGCTGAATTAAAATGGTATAAAT

wherein:

a. N₁ . . . N₂₁ was the small nucleic acid molecule sequence, and N₂₂ . . . N₄₂ was the reverse complement sequence of the small nucleic acid molecule sequence;

b. if the plasmid was required to express shRNA-miRs of multiple genes, each gene corresponded to an shRNA-miR expression framework, and they were then seamlessly connected;

c. constitutive shRNA-miR plasmids with different resistance genes were only different in resistance gene, and the other sequences were the same;

d. the base M represented the base A or C, and N represented the base A, T, G, or C;

e. if N1 was the base G, then M1 was the base A; otherwise, M1 was the base C; and

f. the base M1 was complementary to the base M2.

Second nucleic acid molecule (B2M-3′UTR-miRNA-locus/CIITA-3′UTR-miRNA-locus):

(SEQ ID NO. 2)
atTCTAGATACAGTACTTTTGTGTAGTACAACGTACAGTACTTTTGTGT
AGTACAACGTACAGTACTTTTGTGTAGTACAACGTACAGTACTTTTGTG
TAGTACAACGTACAGTACTTTTGTGTAGTACAACGTACAGTACTTTTGT
GTAGTACAACGTACAGTACTTTTGTGTAGTACAACGTACAGTACTTTTG
TGTAGTACAACGTA

1.3 Immune-Related Gene

The immune-related genes selected in this example were B2M (NCBI Gene ID: 567) and CIITA (NCBI Gene ID: 4261), and a second nucleic acid sequence was inserted at the 3′UTR of these two genes.

1.4 Genomic Safe Locus

In this example, the genomic safe locus for gene knock-in was the AAVS1 safe locus. Those skilled in the art can understand that knocking in other genomic safe loci, such as the eGSH safe locus and the H11 safe locus, can also achieve the objects of the present disclosure.

1.5 Inducible Gene Expression System

In this example, the inducible gene expression system was selected from the tet-Off system. Those skilled in the art can understand that the use of a dimer turn-off expression system can also achieve the objects of the present disclosure.

2 Experimental Method

2.1 Gene Knock-In

The inducible gene expression system and the first nucleic acid molecule were knocked into the genomic safe locus of a pluripotent stem cell or a derivative thereof, and the second nucleic acid molecule (SEQ ID NO. 2) was knocked into the 3′UTR of B2M and CIITA genes.

(I) Construction of sgRNA

1. Plasmid

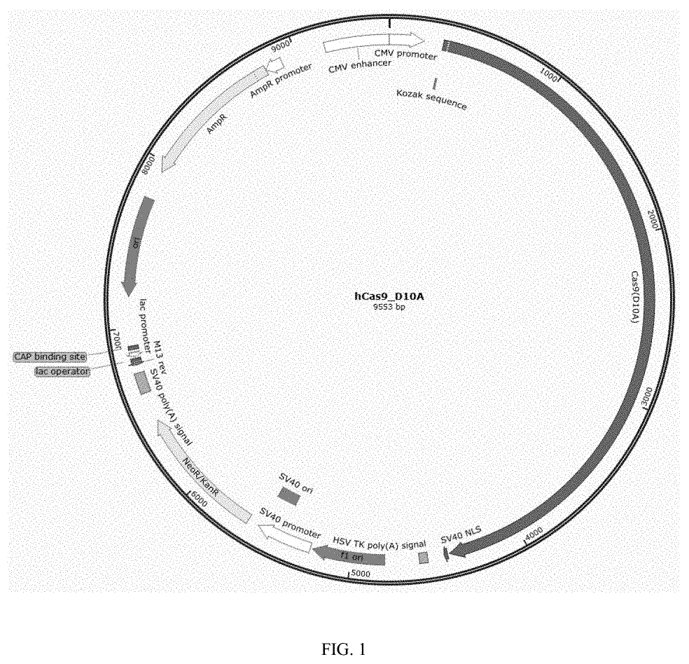

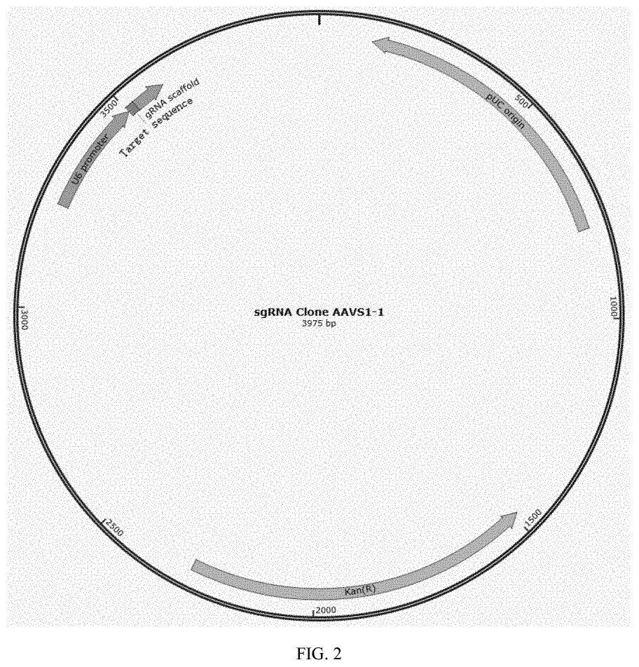

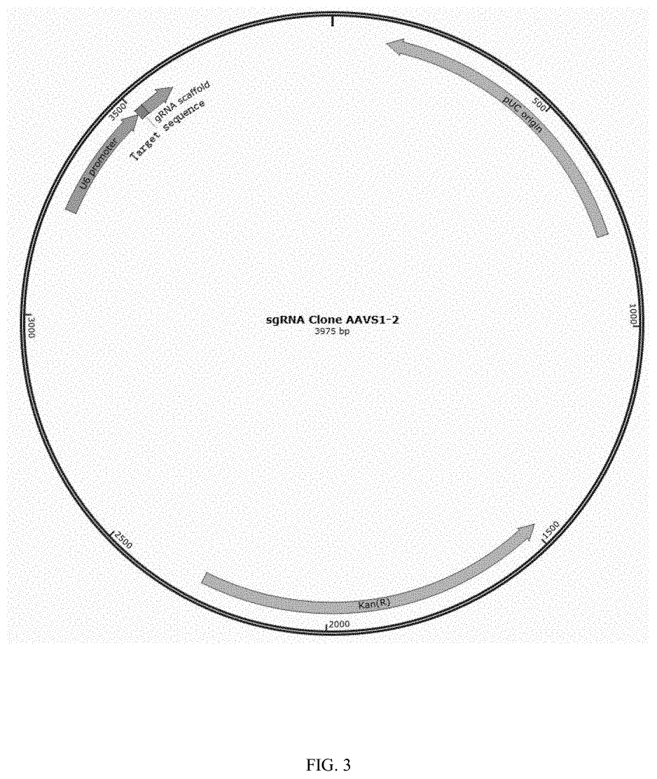

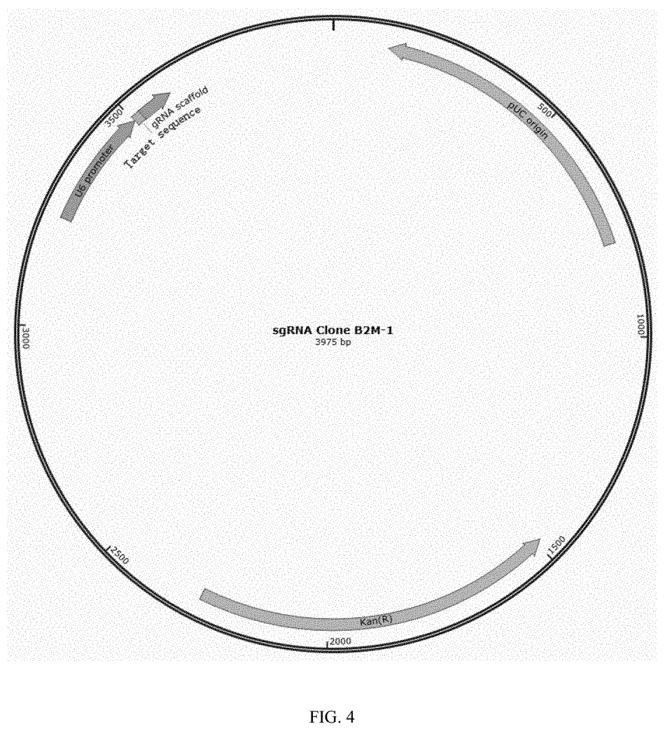

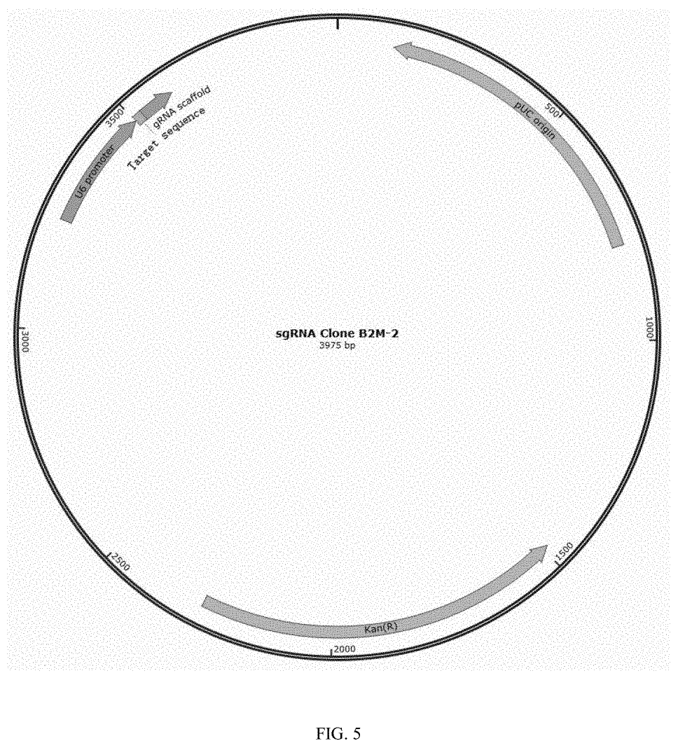

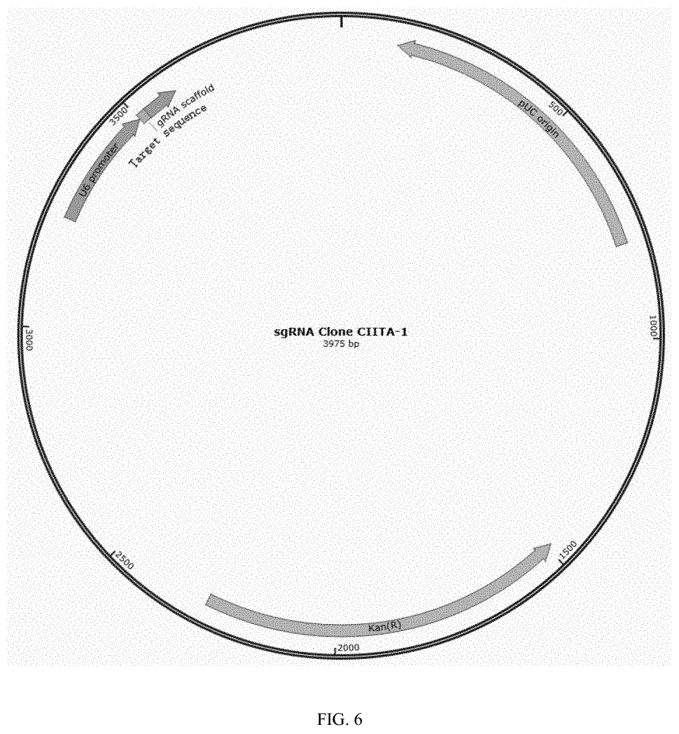

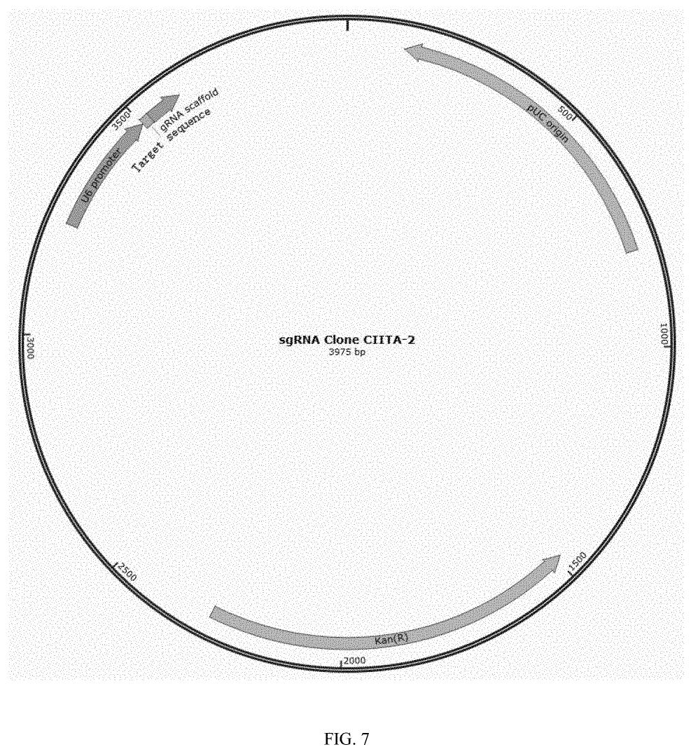

For the knock-in of exogenous genes, Cas9(D10A) plasmid and sgRNA plasmid system were used. The map of Cas9(D10A) plasmid was shown in FIG. 1, the map of the AAVS1 safe locus sgRNA plasmid was shown in FIGS. 2 and 3, the map of B2M gene sgRNA plasmid was shown in FIGS. 4 and 5, and the map of CIITA gene sgRNA plasmid was shown in FIGS. 6 and 7.

2. Homologous Arms

(1). The nucleotide sequences of the AAVS1 homology arms AAVS1-HR-L and AAVS1-HR-R were as shown in SEQ ID NO. 10 and SEQ ID NO. 11, respectively;

(2) the nucleotide sequences of the B2M homology arms B2M-HR-L and B2M-HR-R were as shown in SEQ ID NO. 12 and SEQ ID NO. 13, respectively; and

(3) the nucleotide sequences of the CIITA homology arms CIITA-HR-L and CIITA-HR-R were as shown in SEQ ID NO. 14 and SEQ ID NO. 15, respectively;

3. sgRNA Sequence

sgRNA-AAVS1-1:
(SEQ ID NO. 16)
5′-TATAAGGTGGTCCCAGCTCGGGG-3′;
sgRNA-AAVS1-2:
(SEQ ID NO. 17)
5′-AGGGCCGGTTAATGTGGCTCTGG-3′.
sgRNA-B2M-1:
(SEQ ID NO. 18)
5′-CTCCTGTTATATTCTAGAACAGG-3′;
sgRNA-B2M-2:
(SEQ ID NO. 19)
5′-TTTCAGCATCAATGTACCCTGGG-3′.
sgRNA-CIITA-1:
(SEQ ID NO. 20)
5′-GGCACTCAGAAGACACTGATGGG-3′;
sgRNA-CIITA-2:
(SEQ ID NO. 21)
5′-AAGGTGTCTGGTCGGAGAGCAGG-3′.

4. Plasmid Construction Method

(1) Empty sgRNA vector was digested with the restriction endonuclease BbsI and then recovered.

(2) sgRNA primers (with vector sticky ends) were synthesized.

(3) The primers were diluted with water to 10 μM, a reaction system was prepared and boiled in boiling water for 5 min, and the product was then cooled to room temperature to obtain an annealed product.

Reaction system: upstream primer: 2 μL, downstream primer: 2 μL, and water: 12.8 μL

(4) A DNA ligation reaction kit (TaKaRa, 6022) was used to ligate the vector and the annealed product in the previous steps to obtain a sgRNA plasmid containing the gene target sequence.

(II) Gene Editing Process

1. Single-cell cloning operating steps for AAVS1 gene knock-in

(1) Electrotransfection procedure:

Donor cell preparation: Human pluripotent stem cells

Kit: Human Stem Cell Nucleofector® Kit 1

Instrument: Electrotransfection instrument

Medium: BioCISO

Inducible plasmid: Cas9D10A, sgRNA clone AAVS1-1, sgRNA clone AAVS1-2, AAVS1 neo VectoI, and AAVS1 neo Vector II

(2) The electrotransfected human pluripotent stem cells were screened in a dual-antibiotic medium containing G418 and puro.

(3) Single-cell clones were screened and cultured to obtain a single-cell clone strain.

2. Culture Reagent for AAVS1 Gene Knock-In Single-Cell Clone Strain

(1) Medium: BioCISO+300 μg/ml G418+0.5 μg/ml puro

(It should be placed at room temperature in advance and in the dark for 30-60 minutes until it returned to room temperature. Note: BioCISO should not be preheated at 37° C. to avoid reduced biomolecular activity.)

(2) Matrigel: hESC Grade Matrigel

(Before cell passaging or resuscitation, a Matrigel working solution was added to a cell culture flask or culture dish and shaken until uniform, and it was ensured that the Matrigel completely covered the bottom of the culture flask or culture dish and that the Matrigel at anywhere could not be dried before use. In order to ensure that the cells could better adhere and survive, Matrigel should be placed in an incubator at 37° C. for a coating time, which was not less than 0.5 hours for 1:100×Matrigel and not less than 2 hours for 1:200×Matrigel.)

(3) Digestion solution: EDTA was dissolved with DPBS to a final concentration of 0.5 mM, pH 7.4

(Note: EDTA could not be diluted with water; otherwise, cells would die due to reduced osmotic pressure.)

(4) Cryopreservative fluid: 60% BioCISO+30% ESCs grade FBS+10% DMSO

3. Routine maintenance subculture process

(1) Optimal time for passage and passage ratio

a. Optimal time for passage: When the overall cell confluence reached 80% to 90%.

b. Optimal ratio for passage: 1:4 to 1:7 passage, and the optimal confluence on the next day should be maintained at 20% to 30%.

(2) Passaging process

a. The Matrigel in the coated cell culture flask or cell culture dish was aspirated and discarded in advance, an appropriate amount of medium (BioCISO+300 μg/ml G418+0.5 μg/ml puro) was added, and the flask or dish was placed in an incubator at 37° C., 5% CO₂;

b. when the cells met passage requirements, the medium supernatant was aspirated, and an appropriate amount of 0.5 mM EDTA digestion solution was added to the cell flask or cell dish;

c. the cells were put into an incubator at 37° C., 5% CO₂ and incubated for 5-10 minutes (digested until most of the cells observed under a microscope were shrunk and rounded but not yet floating, then the cells were detached from the wall by gentle pipetting, and the cell suspension was aspirated into a centrifuge tube for centrifugation at 200 g for 5 min);

d. after centrifugation, the supernatant was discarded, the cells were resuspended with a medium, the cells were repeatedly gently pipetted several times until uniformly mixed, and the cells were then transferred to a Matrigel-coated flask or dish prepared in advance;

e. after the cells were transferred to the cell flask or cell dish, the flask or dish was shaken horizontally back and forth and from side to side, and after no abnormality was observed under the microscope, the cells were shaken uniform and placed in an incubator for culturing at 37° C., 5% CO₂; and

f. the next day, the adherence and survival state of the cells were observed, and the medium was aspirated and replaced every day on schedule as normal.

4. Cell Cryopreservation

(1) According to routine passaging operating steps, the cells were digested with 0.5 mM EDTA until most of the cells were shrunk and rounded but not yet floating, the cells were gently pipetted, and the cell suspension was collected and centrifuged at 200 g for 5 minutes; then the supernatant was discarded, an appropriate amount of cryopreservative fluid was added to resuspend the cells, and the cells were transferred to cryopreservation tubes (it was recommended to cryopreserve one tube for a 6-well plate confluence of 80%, and the volume of the cryopreservative fluid was 0.5 ml/tube);

(2) the cryopreservation tubes were placed in a programmed cooling box and immediately placed at −80° C. overnight (it was necessary to ensure that the temperature of the cryopreservation tubes decreased by 1° C. per minute); and

(3) the next day, the cells were immediately transferred into liquid nitrogen.

5. Cell Resuscitation

(1) A Matrigel-coated cell flask or cell dish was prepared in advance; and before cell resuscitation, the Matrigel was aspirated, an appropriate amount of BioCISO was added to the cell flask or cell dish, and the cell flask or cell dish was incubated in an incubator at 37° C., 5% CO₂;

(2) the cryopreservation tubes were quickly taken out from the liquid nitrogen, immediately placed in a water bath at 37° C. and quickly shaken to quickly thaw the cells; and upon careful observation, when ice crystals disappeared completely, shaking was stopped, and the cells were transferred to a biological safety cabinet;

(3) 10 ml DMEM/F12 (1:1) basal medium was added to a 15 ml centrifuge tube in advance and equilibrated to room temperature, 1 ml DMEM/F12 (1:1) was pipetted by a Pasteur pipette, slowly added to the cryopreservation tube, and gently mixed, and the cell suspension was transferred to the prepared 15 ml centrifuge tube containing DMEM/F12 (1:1), followed by centrifugation at 200 g for 5 min;

(4) the supernatant was carefully discarded, an appropriate amount of BioCISO was added, the cells were gently mixed until uniform and seeded in the cell flask or cell dish prepared in advance; the flask or dish was shaken horizontally back and forth and from side to side, and after no abnormality was observed under the microscope, the cells were shaken uniform and placed in an incubator for culturing at 37° C., 5% CO₂; and

(5) the next day, the adherence and survival state of the cells were observed, and the medium was replaced every day on schedule as normal. If the adherence was good, BioCISO was replaced with BioCISO+300 μg/ml G418+0.5 μg/ml puro.

(III) Detection Method of Gene Knock-In

1. Detection of Single-Cell Clone AAVS1 Gene Knock-In
(1) Instructions for AAVS1 gene knock-in detection

a. Test objective: gene-knock-in-treated cells were detected by PCR to test whether the cells were homozygous. Since the two donor fragments only had differences in terms of resistance gene sequence, in order to determine whether the cells were homozygous (the two chromosomes were respectively knocked in with donor fragments of different resistance genes), it was necessary to detect whether the genome of the cells contained the donor fragments of the two resistance genes, and only cells with dual knock-in were likely to be correct homozygotes.

b. Test method: Firstly, a primer was designed inside the resistance gene of the donor plasmid, and another primer was then designed in the insertion locus of the genome (close to the recombination arm). If the donor fragments could be inserted correctly in the genome, there would be bands of interest; otherwise, no bands of interest would appear).

c. The primer sequences and PCR protocols in the test scheme were as shown in Table 1:

TABLE 1
Primer sequences and PCR protocols in the test scheme

| | | | | | PCR |
|---|---|---|---|---|---|
| | | | Sequence | Product | Reaction |
| No. | Primer | Sequence (5′→3′) | number | (bp) | conditions |
| 1 | F1 | CCATAGCTCAGTC | SEQ ID NO. 22 | 2029 | Annealing |
| | | TGGTCTATC | | | at 58° C., |
| 2 | R1 | CTCTTCGTCCAGA | SEQ ID NO. 23 | | extension |
| | | TCATCCTGA | | | for 2 min |
| 3 | F1 | CCATAGCTCAGTC | SEQ ID NO. 22 | 1740 | 10 sec, and |
| | | TGGTCTATC | | | 30 cycles |
| 4 | R2 | CACACCTTGCCGA | SEQ ID NO. 24 | | |
| | | TGTCGAG | | | |

The detection method for the second nucleic acid molecule knock-in at the 3′UTR of B2M and CIITA genes was the same as the detection principle for AAVS1, and the PCR detection conditions were as follows:

TABLE 2
Primer sequences and PCR protocols in the test scheme (detection of
knock-in at the B2M locus)

| | | | | | PCR |
|---|---|---|---|---|---|
| | Primer | | Sequence | Product | Reaction |
| No. | abbreviation | Sequence (5′→3′) | number | (bp) | conditions |
| 1 | F2 | GCACTGAACGAA | SEQ ID NO. 25 | 1994 | Annealing at |
| | | CATCTCAAGAAG | | | 58° C., |
| 2 | R1 | CTCTTCGTCCAGA | SEQ ID NO. 23 | | extension for 2 |
| | | TCATCCTGA | | | min, and 30 |
| 3 | F2 | GCACTGAACGAA | SEQ ID NO. 25 | 1705 | cycles |
| | | CATCTCAAGAAG | | | |
| 4 | R2 | CACACCTTGCCGA | SEQ ID NO. 24 | | |
| | | TGTCGAG | | | |

TABLE 3
Primer sequences and PCR protocols in the test scheme (detection of
knock-in at the CIITA locus)

| | | | | | PCR |
|---|---|---|---|---|---|
| | Primer | | Sequence | Product | Reaction |
| No. | abbreviation | Sequence (5′→3′) | number | (bp) | conditions |
| 1 | F3 | TGCTCCGGGTTTG | SEQ ID NO. 26 | 1952 | Annealing at |
| | | TCTCAGATG | | | 58° C., |
| 2 | R1 | CTCTTCGTCCAGA | SEQ ID NO. 23 | | extension for |
| | | TCATCCTGA | | | 2 min, and |
| 3 | F3 | TGCTCCGGGTTTG | SEQ ID NO. 26 | 1663 | 30 cycles |
| | | TCTCAGATG | | | |
| 4 | R2 | CACACCTTGCCGA | SEQ ID NO. 24 | | |
| | | TGTCGAG | | | |

2.2 Detection of Allogeneic Immunological Compatibility Effect of Stem Cells

2.2.1 Preparation of Effector Cells

Blood was drawn from a volunteer to isolate T cells and NK cells. Effector cells and immunologically compatible pluripotent stem cells were derived from different people.

1) T cell isolation: Human peripheral blood mononuclear cells (PBMCs) were isolated using Ficoll-hypaque density gradient centrifugation, and T cells were isolated using Dynabeads™ CD3 kit (Invitrogen™, Cat. No. 11151D). The cells were resuspended in RPMI1640 medium containing 10% FBS, and the cells were counted by trypan blue staining and concentrated to 1×10⁷ cells/mL.

2) NK cell isolation: NK cells were sorted and isolated using MagniSort™ Human NK cell Enrichment Kit (Invitrogen™, Cat. No. 8804-6819-74). The cells were resuspended in RPMI1640 medium containing 10% FBS, and the cells were counted by trypan blue staining and concentrated to 1×10⁷ cells/mL.

2.2.2 Preparation of Target Cells

Embryoid body cells prepared from PSCs were taken, digested and resuspended, and the cells were counted by trypan blue staining and prepared into a cell suspension of 1×10⁷ cells/mL.

2.2.3 The ⁵¹Cr Release Assay

When normal cells came into contact with T/NK cells (allogeneic), T/NK attacked the normal cells and caused cell lysis and death. However, if there was good immunological compatibility, no attack by T/NK would occur, that is, immune escape. Therefore, the detection of the amount of ⁵¹Cr in the medium could reflect immunological compatibility. The less the amount of ⁵¹Cr released into the detection medium, the better the immunological compatibility.

Cell-mediated cytotoxicity was quantitatively detected, wherein target cells were labeled with the radioisotope ⁵¹Cr and co-incubated with effector molecules or cells, and the cytotoxic activity was determined according to the radiation pulse count (cpm) of ⁵¹Cr released by target cell lysis.

1) The target cells were labeled with 100 μCi (Ci, unit of radioactivity) Na⁵¹CrO₄ at 37° C. for 120 min and shaken every 15 min; after labeling, centrifugation was further carried out 5 times with a washing solution; and finally, the cells were resuspended in a culture solution to 1×10⁶ cells/mL for use.

2) The target cells and T/NK cells were added to a 96-well culture plate, wherein 100 μl of target cells (2.5×10³ cells) and 100 μl of effector cells (E/T=1:2, 1:5, or 1:10, E/T was the ratio of target cells to effector cells (T/NK)), and a natural release control well (100 μl target cells+100 μl medium) and a maximum release well (100 μl target cells+100 μl 2% SDS) were established. They were placed at 37° C., 5% CO₂ for 4 h for incubation. After they were taken out, the supernatant was aspirated from each well with a pipette, then centrifuged, and 100 μl of supernatant was taken to measure the cpm value with a γ counter.

Note: Generally, the natural release rate of ⁵¹Cr was required to be less than 10%.

3) Calculation of results: The natural release rate of ⁵¹Cr and the activity of T/NK cells were calculated according to formulas:

$${}^{S 1} Cr natural release rate ( \% ) = \frac{cpm value of natural release control well}{cpm value of maximal release control well} \times 100 \%$$

$$Activity of T / NK ( \% ) = \frac{\begin{matrix}cpm value of experimental well - \\ cpm value of natural release control well\end{matrix}}{\begin{matrix}cpm value of maximal release control well - \\ cpm value of natural release control well\end{matrix}} \times 100 \%$$

2.2.4 CFSE Test Assay

The fluorescent dye CFSE, also known as CFDA SE (5,6-carboxyfluorescein diacetate, succinimidyl ester, hydroxyfluorescein diacetate succinimidyl ester), was a fluorescent dye that could penetrate cell membranes and could be detected by flow cytometry.

1) A CFSE working solution (with a final concentration of 5 μmol/L CFSE) was added to the target cells, and the cells were incubated at 37° C., 5% CO₂ for 10 min, and washed twice. After trypan blue staining and counting, the cells were resuspended in a medium and prepared into 1×10⁶ cells/mL for later use.

2) The target cells and the effector cells were added to 5 ml flow tubes, wherein 100 μl of target cells (1×10⁵ ml⁻¹) and effector cells (E/T=1:2, 1:5, or 1:10, E/T is the ratio of target cells to effector cells (T)) were added to each tube, and the flow tube in which only target cells were added was used as a control. The effector cells and the target cells in the flow tubes were gently mixed until uniform. PI was added, and they were placed at 37° C., 5% CO₂ for 4 h for incubation. The percentage of CFSE⁺PI⁺ cells (dead target cells) was detected by flow cytometry.

Target cell death rate (%)=death rate of target cells stimulated with T cells (%)−natural death rate of target cells (%)

2.2.5 Analysis of CD₁₀₇ₐ Expression in NK Cells by Flow Cytometer (FCM)

When the NK cells killed the target cells, CD₁₀₇ₐ molecules were transported to the surface of the cell membrane, and NK cells with positive CD₁₀₇ₐ molecule expression could represent NK cells with killing activity.

1) The effector cells and the target cells were mixed at a certain ratio (E/NK=3:1, 1:1, or 1:3, E/NK was the ratio of target cells to effector cells (NK)), placed in culture wells, and incubated at 37° C., 5% CO₂ for 2 h, monensin (2 μmol/L) was added for continued incubation for 3.5 h, and PE-Cy5-CD₁₀₇ₐ and FITC-CD56 antibodies were then added for incubation for 30 min. After washing 3× with PBS buffer, the cells were fixed with 200 μL of 1% paraformaldehyde for flow analysis. The effector cells were simultaneously stimulated with only PMA (2.5 μg/mL) and ionomycin (0.5 μg/mL), as a positive control.

Note: The natural expression frequency of CD₁₀₇ₐ on the surface of the NK cell membrane was very low, about 1.2% to 5.8%.

2) Calculation of Results

NK cell cytotoxicity=CD₁₀₇ₐ positive rate upon stimulation by target cells (%) −CD₁₀₇ₐ natural expression rate (%)

2.2.6 MTT Experiment for Detecting Cell Viability

After digestion and cell counting, the cells were blown uniform with a corresponding medium and plated into a 96-well plate in which each well was seeded with 3000 cells, with 5 duplicate wells, the wells were then supplemented with the corresponding medium to a final volume of 150 uL, the corresponding medium was changed every day, the cells were placed in an incubator at 37° C., 5% CO₂ for 72 h, and the MTT value was measured.

3 Experimental Scheme

Experimental Scheme I:

The specific experimental groupings were as shown in Table 4, and “+” indicated knock-in of the corresponding item in the genome. In the scheme:

B2M-3′UTR-miRNA-locus or CIITA-3′UTR-miRNA-locus, i.e. the second nucleic acid molecule (SEQ ID NO. 2), was knocked into the 3′UTR regions of B2M and CIITA genes, respectively.

B2M/CIITA-3′UTR-shRNA was the shRNA expression framework of the small nucleic acid molecule, i.e., the first nucleic acid molecule, which specifically targeted the transcript of the second nucleic acid molecule in the 3′UTR regions of the B2M gene and CIITA gene, and the knock-in locus was the genomic safe locus AAVS1.

B2M/CIITA-3′UTR-shRNA-miR was the shRNA-miR expression framework of the small nucleic acid molecule, i.e., the first nucleic acid molecule, which targeted the transcript of the second nucleic acid molecule in the 3′UTR regions of the B2M gene and CIITA gene, and the knock-in locus was the genomic safe locus AAVS1.

CD47 represented the CD47 expression sequence, and the knock-in locus thereof was the genomic safe locus AAVS1.

TABLE 4
Constitutive expression experiment scheme

| | Experimental group | | | | |
|---|---|---|---|---|---|
| | A1 | A2 | A3 | A4 | A5 |
| B2M-3′UTR-miRNA-locus | | + | + | + | + |
| CIITA-3′UTR-miRNA-locus | | + | + | + | + |
| B2M/CIITA-3′UTR-shRNA | | + | | + | |
| B2M/CIITA-3′UTR-shRNA-miR | | | + | | + |
| CD47 | | | | + | + |

Experimental Scheme II:

The specific experimental groupings were as shown in Table 5, and “+” indicated knock-in of the corresponding item in the genome. In the scheme:

B2M-3′UTR-miRNA-locus or CIITA-3′UTR-miRNA-locus, i.e. the second nucleic acid molecule (SEQ ID NO. 2), was knocked into the 3′UTR regions of B2M and CIITA genes, respectively.

B2M/CIITA-3′UTR-shRNA was the shRNA expression framework of the small nucleic acid molecule, i.e., the first nucleic acid molecule, which specifically targeted the transcript of the second nucleic acid molecule in the 3′UTR regions of the B2M gene and CIITA gene, and the knock-in locus was the genomic safe locus AAVS1.

B2M/CIITA-3′UTR-shRNA-miR was the shRNA-miR expression framework of the small nucleic acid molecule, i.e., the first nucleic acid molecule, which targeted the transcript of the second nucleic acid molecule in the 3′UTR regions of the B2M gene and CIITA gene, and the knock-in locus was the genomic safe locus AAVS1.

CD47 represented CD47 expression sequence, and the knock-in locus thereof was the genomic safe locus AAVS1.

The knock-in locus for the Tet-Off system was the genomic safe locus AAVS1, and it was used to regulate the expression of shRNA or shRNA-miR and CD47.

TABLE 5
Inducible expression experiment scheme

| | Experimental group | | | | |
|---|---|---|---|---|---|
| | B1 | B2 | B3 | B4 | B5 |
| B2M-3′UTR-miRNA-locus | | + | + | + | + |
| CIITA-3′UTR-miRNA-locus | | + | + | + | + |
| B2M/CIITA-3′UTR-shRNA | | + | | + | |
| B2M/CIITA-3′UTR-shRNA-miR | | | + | | + |
| CD47 | | | | + | + |
| Tet-Off system | | + | + | + | + |

Experimental Operation of Each Experimental Group

1. Construction of expression plasmid

KI (Knock-in) plasmid construction method:

a. Acquisition of basic backbone of the plasmid: Primers were designed, an Amp(R)-pUC origin fragment was obtained from pUC18 (Takara, Code No. 3218) plasmid by PCR, and the product was then recovered.

b. Acquisition of recombination arms: Primers were designed, the genomic DNA of a human cell was taken as a template, AAVS1-HR-L (SEQ ID NO. 10), AAVS1-HR-R (SEQ ID NO. 11), B2M-HR-L (SEQ ID NO. 12), B2M-HR-R (SEQ ID NO. 13), CIITA-HR-L (SEQ ID NO. 14) and CIITA-HR-R (SEQ ID NO. 15) fragments were amplified, and these product were then recovered.

c. Acquisition of other plasmid elements: Primers were designed, plasmids containing the plasmid elements were directly subcloned, and a product was then recovered.

d. Plasmid assembly: The various products obtained in the previous steps were ligated into large fragments by overlap PCR, and finally, the large fragments were ligated into a circular plasmid by recombination using a recombinase (Nanjing Vazyme Biotech, C113-01).

2. The operation of inserting the molecules of each group into the KI plasmid was as follows:

(1) Group A1: Blank control group without any treatment.

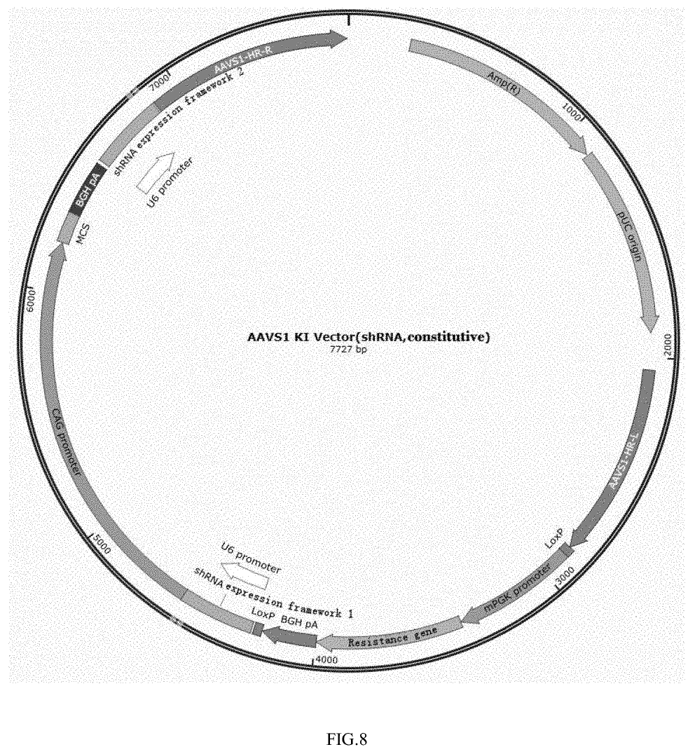

(2) Group A2: The small nucleic acid molecule sequence SEQ ID NO. 1 was put into shRNA expression framework 1 of AAVS1 KI Vector(shRNA,constitutive) plasmid (FIG. 8);

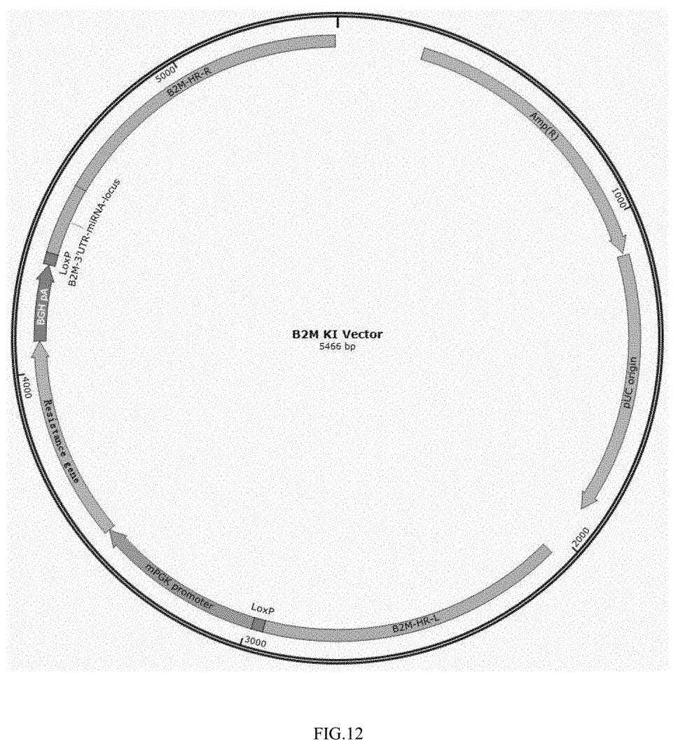

B2M-3′UTR-miRNA-locus was put into B2M KI Vector (as shown in FIG. 12);

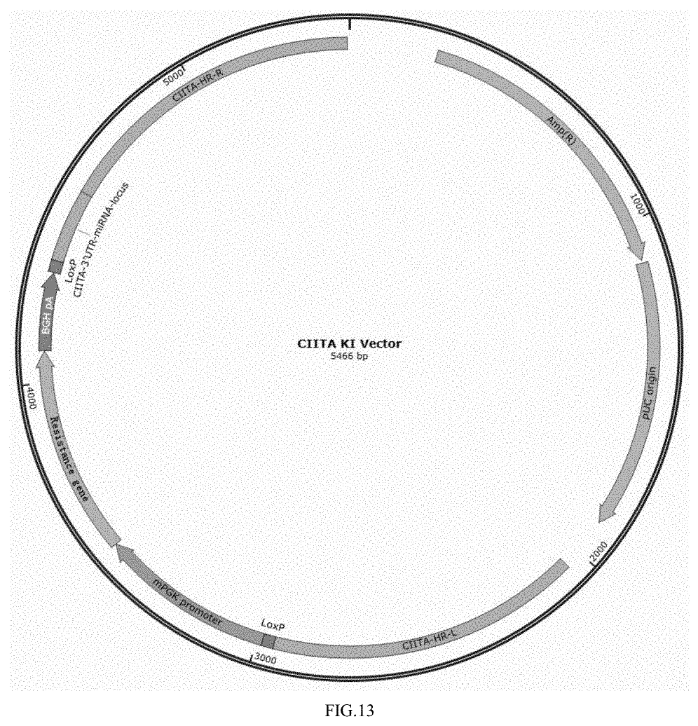

and CIITA-3′UTR-miRNA-locus was put into CIITA KI Vector (as shown in FIG. 13).

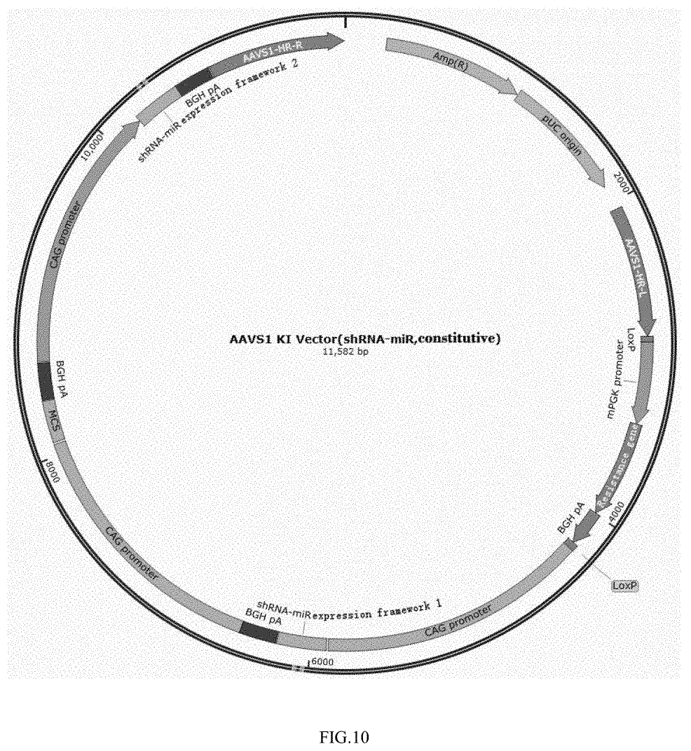

(3) Group A3: The small nucleic acid molecule sequence SEQ ID NO. 1 was put into shRNA-miR expression framework 1 of AAVS1 KI Vector(shRNA-miR,constitutive) plasmid (FIG. 10);

B2M-3′UTR-miRNA-locus was put into B2M KI Vector; and

CIITA-3′UTR-miRNA-locus was put into CIITA KI Vector.

(4) Group A4: CD47 gene sequence was put into MCS of AAVS1 KI Vector(shRNA,constitutive) plasmid, and the small nucleic acid molecule sequence SEQ ID NO. 1 was put into shRNA expression framework 1;

B2M-3′UTR-miRNA-locus was put into B2M KI Vector (as shown in FIG. 12);

and CIITA-3′UTR-miRNA-locus was put into CIITA KI Vector (as shown in FIG. 13).

(5) Group A5: CD47 gene sequence was put into MCS of AAVS1 KI Vector(shRNA-miR,constitutive) plasmid, and the small nucleic acid molecule sequence SEQ ID NO. 1 was put into shRNA-miR expression framework 1;

B2M-3′UTR-miRNA-locus was put into B2M KI Vector; and

CIITA-3′UTR-miRNA-locus was put into CIITA KI Vector.

(6) Group B1: Blank control group without any treatment.

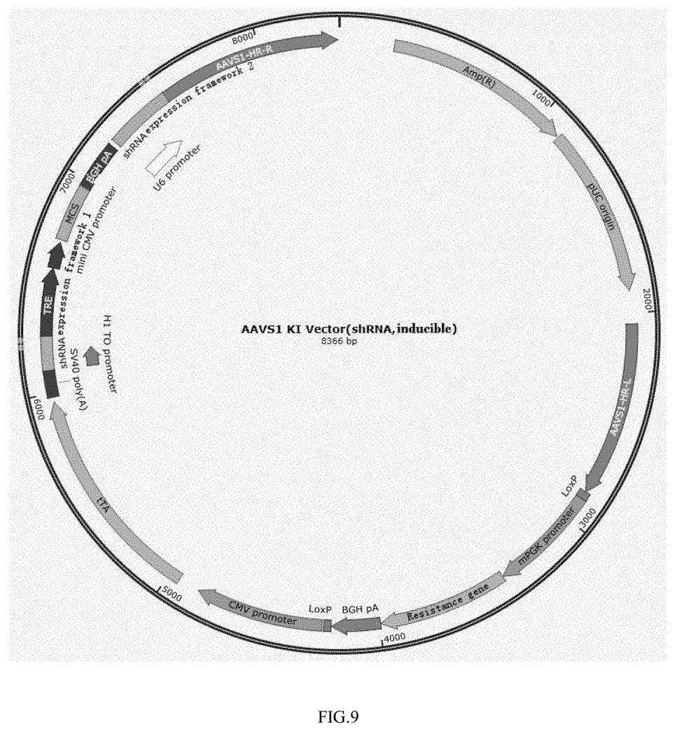

(7) Group B2: The small nucleic acid molecule sequence SEQ ID NO. 1 was put into shRNA expression framework 1 of AAVS1 KI Vector(shRNA,inducible) plasmid (FIG. 9);

B2M-3′UTR-miRNA-locus was put into B2M KI Vector; and

CIITA-3′UTR-miRNA-locus was put into CIITA KI Vector.

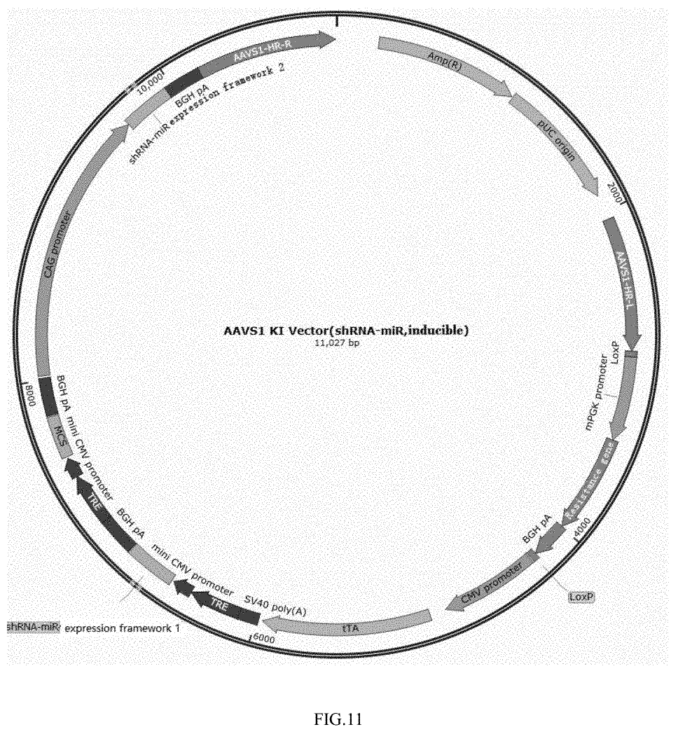

(8) Group B3: The small nucleic acid molecule sequence SEQ ID NO. 1 was put into shRNA-miR expression framework 1 of AAVS1 KI Vector(shRNA-miR,inducible) plasmid (FIG. 11);

B2M-3′UTR-miRNA-locus was put into B2M KI Vector; and

CIITA-3′UTR-miRNA-locus was put into CIITA KI Vector.

(9) Group B4: CD47 gene sequence was put into MCS of AAVS1 KI Vector(shRNA,inducible) plasmid, and the small nucleic acid molecule sequence SEQ ID NO. 1 was put into shRNA expression framework 1;

B2M-3′UTR-miRNA-locus was put into B2M KI Vector; and

CIITA-3′UTR-miRNA-locus was put into CIITA KI Vector.

(10) Group B5: CD47 gene sequence was put into MCS of AAVS1 KI Vector(shRNA-miR,inducible) plasmid, and the small nucleic acid molecule sequence SEQ ID NO. 1 was put into shRNA-miR expression framework 1;

B2M-3′UTR-miRNA-locus was put into B2M KI Vector; and

CIITA-3′UTR-miRNA-locus was put into CIITA KI Vector.

4. Experiment Results

4.1 Detection of the immunological compatibility effect of engineered stem cells or derivative thereof by the ⁵¹Cr release assay

According to the experimental schemes in Tables 4 and 5, hPSC-derived EBs were engineered, and the ⁵¹Cr release assay was used to detect the effect of immunological compatibility between the engineered EB spheres and T cells:

1. The EB sphere immunologically compatible cells were digested into individual cells as target cells;

2. ⁵¹Cr-labeled target cells and T cells were added to a 96-well culture plate at a ratio of 1:5 for post-reaction detection; and

3. the cell-specific ⁵¹Cr release rate of the EB sphere immunologically compatible cells was detected according to the ⁵¹Cr release assay, and the results are as shown in Table 6.

TABLE 6
Cell-specific ⁵¹Cr release rates of EB sphere
immunologically compatible cells

| Group | | Mean ⁵¹Cr release rate (%) | Deviation (±) |
|---|---|---|---|
| No Dox | A1 | 56.85 | 1.73 |
| | A2 | 41.08 | 1.58 |
| | A3 | 40.38 | 1.10 |
| | A4 | 34.20 | 0.91 |
| | A5 | 36.13 | 1.35 |
| | B2 | 40.18 | 0.71 |
| | B3 | 39.39 | 1.23 |
| | B4 | 35.41 | 2.27 |
| | B5 | 35.31 | 1.74 |
| +Dox | A1 | 55.51 | 3.58 |
| | A2 | 40.65 | 1.46 |
| | A3 | 40.02 | 2.09 |
| | A4 | 34.50 | 0.20 |
| | A5 | 35.07 | 0.53 |
| | B2 | 56.78 | 2.01 |
| | B3 | 56.49 | 2.55 |
| | B4 | 55.31 | 0.62 |
| | B5 | 57.43 | 1.75 |

It can be seen from Table 6 that the immunological compatibility effect of the hPSC-derived EB sphere cells that we prepared was significant. In addition, after 6 μM of Dox was added to the medium to treat the cells for 48 h, the inducible expression groups (B2-B5) restored antigen-presenting ability and were presented in an immunologically non-compatible state, thus realizing the reversible regulation of the immunological compatibility of the cells.

⁵¹Cr release assay was further used to detect the effect of immunological compatibility between the engineered EB sphere immunologically compatible cells and NK cells:

1. The EB sphere immunologically compatible cells were digested into individual cells as target cells;

2. ⁵¹Cr-labeled target cells and NK cells were added to a 96-well culture plate at a ratio of 1:5 for post-reaction detection; and

3. the cell-specific ⁵¹Cr release rate of the EB sphere immunologically compatible cells was detected according to the ⁵¹Cr release assay, and the results are as shown in Table 7.

TABLE 7
Cell-specific ⁵¹Cr release rates of EB sphere
immunologically compatible cells

| Group | | Mean ⁵¹Cr release rate (%) | Deviation (±) |
|---|---|---|---|
| No Dox | A1 | 53.22 | 0.84 |
| | A2 | 33.38 | 0.53 |
| | A3 | 34.20 | 1.44 |
| | A4 | 31.03 | 0.37 |
| | A5 | 30.16 | 0.31 |
| | B2 | 32.17 | 1.34 |
| | B3 | 33.16 | 1.92 |
| | B4 | 30.74 | 2.01 |
| | B5 | 30.34 | 1.80 |
| +Dox | A1 | 54.05 | 2.49 |
| | A2 | 34.72 | 1.76 |
| | A3 | 34.59 | 0.50 |
| | A4 | 31.79 | 0.90 |
| | A5 | 31.83 | 0.72 |
| | B2 | 51.86 | 0.71 |
| | B3 | 54.46 | 2.35 |
| | B4 | 55.00 | 1.34 |
| | B5 | 53.60 | 1.68 |

It can be seen from Table 7 that the immunological compatibility effect of the hPSC-derived EB sphere cells that we prepared was significant. In addition, after 6 μM of Dox was added to the medium to treat the cells for 48 h, the inducible expression groups (B2-B5) restored antigen-presenting ability and were presented in an immunologically non-compatible state, thus realizing the reversible regulation of the immunological compatibility of the cells.

4.2 Detection of the Effect of Exosomes (Secreted by EB Sphere Immunologically Compatible Cells) on Other Cells in Terms of Immune Escape Development by CFSE Assay

1. According to the experimental schemes in Tables 4 and 5, hPSC-derived EBs were engineered, the obtained EB sphere immunologically compatible cells were cultured, and the culture supernatant was collected to extract exosomes;

2. the exosomes were added to non-engineered EB sphere immunologically non-compatible cells and EB sphere immunologically non-compatible cells engineered with B2M&CIITA-3′UTR-miRNA-locus (engineering scheme: only B2M-3′UTR-miRNA-locus or CIITA-3′UTR-miRNA-locus, i.e. the second nucleic acid molecule (SEQ ID NO. 2), was knocked into the 3′UTR regions of the B2M and CIITA genes, respectively.), and the cells were cultured for 72 h and digested into individual cells as target cells;

3. CFSE-labeled target cells and T cells were added to a 5 ml flow tube at a ratio of 1:5 for a reaction and then detected; and

4. the percentage of CFSE+PI+ cells (dead target cells) was detected according to CFSE test assay, and the results are shown in Table 8.

TABLE 8
CFSE test results

| | | | Mean death | |
|---|---|---|---|---|
| | | | rate of | |
| | | | target cell | Deviation |
| Group | | | (%) | (±) |
| Non-engineered | No Dox | N | 60.76 | 2.26 |
| EB sphere | | A2 | 58.47 | 2.60 |
| immunologically | | A3 | 61.29 | 2.85 |
| non-compatible | | A4 | 59.66 | 2.59 |
| cells | | A5 | 59.92 | 0.82 |
| | | B2 | 58.74 | 0.90 |
| | | B3 | 61.13 | 2.81 |
| | | B4 | 62.26 | 1.97 |
| | | B5 | 61.65 | 1.39 |
| | +Dox | N | 60.04 | 0.42 |
| | | A2 | 60.25 | 2.79 |
| | | A3 | 60.31 | 2.16 |
| | | A4 | 61.34 | 2.05 |
| | | A5 | 60.33 | 1.08 |
| | | B2 | 59.84 | 1.69 |
| | | B3 | 62.31 | 1.78 |
| | | B4 | 62.26 | 1.45 |
| | | B5 | 60.33 | 1.50 |
| EB sphere | No Dox | N | 62.91 | 4.37 |
| immunologically | | A2 | 33.90 | 1.19 |
| non-compatible | | A3 | 33.51 | 0.54 |
| cells | | A4 | 35.07 | 1.39 |
| engineered with | | A5 | 34.88 | 1.02 |
| B2M&CIITA- | | B2 | 33.88 | 0.86 |
| 3′UTR- | | B3 | 33.79 | 1.00 |
| | | B4 | 34.01 | 0.36 |
| | | B5 | 34.01 | 1.00 |
| | +Dox | N | 62.21 | 4.38 |
| | | A2 | 34.93 | 0.77 |
| | | A3 | 34.28 | 1.14 |
| | | A4 | 34.56 | 1.36 |
| | | A5 | 34.56 | 1.09 |
| | | B2 | 62.66 | 3.63 |
| | | B3 | 60.82 | 2.11 |
| | | B4 | 62.93 | 0.93 |
| | | B5 | 63.15 | 0.38 |

It can be seen from Table 8 that when B2M/CIITA-3′UTR-shRNA and B2M/CIITA-3′UTR-shRNA-miR (produced by the cells) reached recipient cells via the exosomes, they only acted on the cells engineered by B2M&CIITA-3′UTR-miRNA-locus knock-in to produce immunological compatibility and did not cause other non-donor cells to produce immunological compatibility effects, that is, the immunological compatibility effect produced by the cells could only act on the donor cells.

4.3 Analysis of CD₁₀₇ₐ Expression in NK Cells by Flow Cytometer (FCM) to Detect the Effect of Exosomes (Secreted by EB Sphere Immunologically Compatible Cells) on Other Cells in Terms of Immune Escape Development

1. According to the experimental schemes in Tables 4 and 5, hPSC-derived EBs were engineered, the obtained EB sphere immunologically compatible cells were cultured, and the culture supernatant was collected to extract exosomes;

2. the exosomes were added to non-engineered EB sphere immunologically non-compatible cells and EB sphere immunologically non-compatible cells engineered with B2M&CIITA-3′UTR-miRNA-locus (engineering scheme: only B2M-3′UTR-miRNA-locus or CIITA-3′UTR-miRNA-locus, i.e. the second nucleic acid molecule (SEQ ID NO. 2), was knocked into the 3′UTR regions of the B2M and CIITA genes, respectively.), and the cells were cultured for 72 h and digested into individual cells as target cells;

3. the target cells and NK cells were added to a 5 ml flow tube at a ratio of 1:1 for a reaction and then detected; and

4. the cytotoxicity of the NK cells was detected according to the expression of CD₁₀₇ₐ in the NK cells, and the results are shown in Table 9.

TABLE 9
Detection results of CD₁₀₇ₐ expression in NK cells

| | | | Mean | |
|---|---|---|---|---|
| | | | cytotoxicity | |
| Group | | | of NK | Deviation |
| | | | cell(%) | (±) |
| Non-engineered | No Dox | N | 55.28 | 1.04 |
| EB sphere | | A2 | 54.12 | 3.62 |
| immunologically | | A3 | 55.88 | 4.61 |
| non-compatible | | A4 | 54.75 | 1.30 |
| cells | | A5 | 54.37 | 0.27 |
| | | B2 | 54.31 | 4.46 |
| | | B3 | 56.42 | 2.42 |
| | | B4 | 54.78 | 1.34 |
| | | B5 | 56.87 | 0.87 |
| | +Dox | N | 55.66 | 2.81 |
| | | A2 | 54.78 | 4.36 |
| | | A3 | 52.10 | 1.88 |
| | | A4 | 56.96 | 3.00 |
| | | A5 | 55.06 | 1.41 |
| | | B2 | 54.90 | 0.86 |
| | | B3 | 55.77 | 0.65 |
| | | B4 | 56.66 | 0.80 |
| | | B5 | 54.65 | 1.08 |
| EB sphere | No Dox | N | 56.43 | 3.12 |
| immunologically | | A2 | 30.50 | 0.71 |
| non-compatible | | A3 | 30.64 | 0.81 |
| cells | | A4 | 30.15 | 0.92 |
| engineered with | | A5 | 30.59 | 1.20 |
| B2M&CIITA- | | B2 | 29.77 | 0.46 |
| 3′UTR- | | B3 | 30.64 | 1.24 |
| miRNA-locus | | B4 | 30.35 | 0.81 |
| | | B5 | 30.60 | 1.08 |
| | +Dox | N | 54.91 | 4.10 |
| | | A2 | 30.52 | 1.23 |
| | | A3 | 30.57 | 1.03 |
| | | A4 | 30.40 | 1.30 |
| | | A5 | 31.46 | 0.45 |
| | | B2 | 55.90 | 1.77 |
| | | B3 | 56.24 | 3.58 |
| | | B4 | 56.94 | 0.73 |
| | | B5 | 57.83 | 3.20 |

It can be seen from Table 9 that when B2M/CIITA-3′UTR-shRNA and B2M/CIITA-3′UTR-shRNA-miR produced by the cells reached recipient cells via the exosomes, they only acted on the cells engineered by B2M&CIITA-3′UTR-miRNA-locus knock-in to produce immunological compatibility and did not cause other non-donor cells to produce immunological compatibility effects, that is, the immunological compatibility effect produced by the cells could only act on the donor cells.

4.4 Immunological Compatibility Effects of Different Pluripotent Stem Cells or Derivatives Thereof

hPSCs, hPSCs-MSCs, NSCs, and EB cells were respectively engineered according to the experimental scheme of Group B4 in Table 5. These engineered cells were respectively digested into individual cells as target cells, and the ⁵¹Cr release assay was used to detect the immunological compatibility effect of the target cells:

1. ⁵¹Cr-labeled target cells and T cells were added to a 96-well culture plate at a ratio of 1:5 for post-reaction detection; and

2. the cell-specific ⁵¹Cr release rate of the target cells was detected according to the ⁵¹Cr release assay, and the results are as shown in Table 10.

TABLE 10
⁵¹Cr release rates of hPSC-MSC, NSC and EB
immunologically compatible cells

| | Group | Mean ⁵¹Cr release rate (%) | Deviation (±) |
|---|---|---|---|
| hPSCs | 1 | 52.99 | 1.55 |
| | 2 | 30.10 | 0.57 |
| | 3 | 53.48 | 0.69 |
| MSCs | 1 | 50.54 | 0.73 |
| | 2 | 28.36 | 0.69 |
| | 3 | 49.33 | 0.63 |
| NSCs | 1 | 57.24 | 0.40 |
| | 2 | 31.10 | 1.01 |
| | 3 | 57.22 | 0.87 |
| EBs | 1 | 59.87 | 0.94 |
| | 2 | 30.36 | 0.62 |
| | 3 | 58.34 | 0.55 |

Note: Group 1 was a control group (non-engineered cell group); Group 2 was a constructed immunologically compatible cell group (scheme B4); and Group 3 was an immunologically compatible cell group treated with an inducer (Dox).

It can be seen from Table 10 that the immunological compatibility effects of the hPSCs and hPSC-derived derivatives (hPSCs-MSCs, NSCs, and EBs) that we prepared were significant, and after being treated with the inducer (Dox), these cells could all restore antigen-presenting ability and achieve reversibility of immunological compatibility.

1. A pluripotent stem cell or a derivative thereof, wherein a first nucleic acid molecule is introduced into the genome of the pluripotent stem cell or the derivative thereof; and a second nucleic acid molecule is introduced into a 3′UTR region of an immune response-related gene in the pluripotent stem cell or the derivative thereof;
the first nucleic acid molecule encodes a small nucleic acid molecule that mediates RNA interference, the small nucleic acid molecule specifically targets a transcript of the second nucleic acid molecule, and the small nucleic acid molecule does not target any other mRNA or lncRNA of the pluripotent stem cell or the derivative thereof.

2. The pluripotent stem cell or the derivative thereof according to claim 1, wherein an inducible gene expression system is further introduced into the genome of the pluripotent stem cell or the derivative thereof for regulating the expression of the first nucleic acid molecule.

3. The pluripotent stem cell or the derivative thereof according to claim 1, wherein an expression sequence of CD47 is further introduced into the genome of the pluripotent stem cell or the derivative thereof.

4. The pluripotent stem cell or the derivative thereof according to claim 1, wherein the immune response-related gene includes:
(1) major histocompatibility complex-related genes, including at least one selected from the group consisting of B2M and CIITA; and
(2) major histocompatibility complex genes, including at least one selected from the group consisting of HLA-A, HLA-B, HLA-C, HLA-DRA, HLA-DRB1, HLA-DRB3, HLA-DRB4, HLA-DRB5, HLA-DQA1, HLA-DQB1, HLA-DPA1 and HLA-DPB1.

5. The pluripotent stem cell or the derivative thereof according to claim 1, wherein the small nucleic acid molecule includes short interfering nucleic acid, short interfering RNA, and double-stranded RNA.

6. The pluripotent stem cell or the derivative thereof according to claim 5, wherein the pluripotent stem cell or the derivative thereof is derived from human; and the sequence of the small nucleic acid molecule is a random sequence derived from a non-human species, which does not target any human mRNA or lncRNA.

7. The pluripotent stem cell or the derivative thereof according to claim 6, wherein the sequence of the small nucleic acid molecule is any one of:
5′-TTGTACTACACAAAAGTACTG-3′;
and
5′-TCACAACCTCCTAGAAAGAGTAGA-3′.

8. The pluripotent stem cell or the derivative thereof according to claim 1, wherein the second nucleic acid molecule includes at least 3 repeats of a reverse complement sequence of the small nucleic acid molecule sequence.

9. The pluripotent stem cell or the derivative thereof according to claim 2, wherein the inducible gene expression system includes at least one selected from the group consisting of Tet-Off system and dimer-induced expression system.

10. The pluripotent stem cell or the derivative thereof according to claim 2, wherein introduction loci for the first nucleic acid molecule and the inducible gene expression system are genomic safe loci, preferably at least one selected from the group consisting of the AAVS1 safe locus, the eGSH safe locus, and the H11 safe locus.

11. The pluripotent stem cell or the derivative thereof according to claim 2, wherein the first nucleic acid molecule and the inducible gene expression system are introduced by means of viral vector interference, non-viral vector transfection or gene editing; the second nucleic acid molecule is introduced by means of gene editing; and the gene editing is preferably gene knock-in.

12. The pluripotent stem cell or the derivative thereof according to claim 1, wherein the pluripotent stem cell includes an embryonic stem cell, an embryonic germ cell, an embryonic carcinoma cell, or an induced pluripotent stem cell;
the derivative includes a pluripotent stem cell-derived three-germ-layer-derived organ, tissue or cell; and
the pluripotent stem cell-derived three-germ-layer-derived cell includes mesenchymal stem cells, neural stem or progenitor cells, or other adult stem cells.

13. A method for cellular therapy or organ transplantation, comprising administering a therapeutically effective amount of the pluripotent stem cell or the derivative thereof according to claim 1 to a subject in need thereof.

14. A universal pluripotent stem cell bank generating by the pluripotent stem cell or the derivative thereof according to claim 1.

15. A gene drug carrier, comprising the pluripotent stem cell or the derivative thereof according to claim 1.

16. The pluripotent stem cell or the derivative thereof according to claim 1, wherein the introduction locus for the first nucleic acid molecule is a genomic safe locus, preferably at least one selected om the group consisting of the AAVS1 safe locus, the eGSH safe locus, and the H11 safe locus.

17. The pluripotent stem cell or the derivative thereof according to claim 1, wherein the first nucleic acid molecule is introduced by means of viral vector interference, non-viral vector transfection or gene editing; the second nucleic acid molecule is introduced by means of gene editing; and the gene editing is preferably gene knock-in.

18. The pluripotent stem cell or the derivative thereof according to claim 1, wherein the small nucleic acid molecule is at least one selected from the group consisting of miRNA, shRNA, and shRNA-miR.

19. The pluripotent stem cell or the derivative thereof according to claim 5, wherein the small nucleic acid molecule is derived from Caenorhabditis elegans.

20. The pluripotent stem cell or the derivative thereof according to claim 8, wherein the second nucleic acid molecule includes 6-10 repeats of the reverse complement sequence of the small nucleic acid molecule sequence.


SEQUENCE LISTING (26 sequences)

SEQ ID NO: 1 - DNA, 21 bp, Caenorhabditis elegans
ttgtactacacaaaagtactg

SEQ ID NO: 2 - DNA, 210 bp, Artificial sequence
  synthesized
attctagatacagtacttttgtgtagtacaacgtacagtacttttgtgtagtacaacgtacagtacttttgtgtagtacaacgtacagtacttttgtgtagtacaacgtacagtacttttgtgtagtacaacgtacagtacttttgtgtagtacaacgtacagtacttttgtgtagtacaacgtacagtacttttgtgtagtacaacgta

SEQ ID NO: 3 - DNA, 24 bp, Caenorhabditis elegans
tcacaacctcctagaaagagtaga

SEQ ID NO: 4 - DNA, 234 bp, Artificial sequence
  synthesized
attctagatatctactctttctaggaggttgtgacgtatctactctttctaggaggttgtgacgtatctactctttctaggaggttgtgacgtatctactctttctaggaggttgtgacgtatctactctttctaggaggttgtgacgtatctactctttctaggaggttgtgacgtatctactctttctaggaggttgtgacgtatctactctttctaggaggttgtgacgta

SEQ ID NO: 5 - DNA, 686 bp, Artificial sequence
  synthesized
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggactttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgaggtaggcgtgtacggtgggaggcctatataagcagagctcgtttagtgaaccgtcagatcgcctggagacgccatccacgctgttttgacctccatagaagacaccgggaccgatccagcctgctagcgccacc

SEQ ID NO: 6 - DNA, 9 bp, Artificial sequence
  synthesized
ttcaagaga

SEQ ID NO: 7 - DNA, 119 bp, Artificial sequence
  synthesized
gaggcttcagtactttacagaatcgttgcctgcacatcttggaaacacttgctgggattacttcttcaggttaacccaacagaaggctaaagaaggtatattgctgttgacagtgagcg

SEQ ID NO: 8 - DNA, 19 bp, Artificial sequence
  synthesized
tagtgaagccacagatgta

SEQ ID NO: 9 - DNA, 119 bp, Artificial sequence
  synthesized
tgcctactgcctcggacttcaaggggctactttaggagcaattatcttgtttactaaaactgaataccttgctatctctttgatacatttttacaaagctgaattaaaatggtataaat

SEQ ID NO: 10 - DNA, 804 bp, Artificial sequence
  synthesized
tgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggc

SEQ ID NO: 11 - DNA, 837 bp, Artificial sequence
  synthesized
actagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 12 - DNA, 900 bp, Artificial sequence
  synthesized
cctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagggaaagaagaatcctacagggtcatgttcccttctcctgtggagtggcatgaagaaggtgtatggccccaggtatggccatattactgaccctctacagagagggcaaaggaactgccagtatggtattgcaggataaaggcaggtggttacccacattacctgcaaggctttgatctttcttctgccatttccacattggacatctctgctgaggagagaaaatgaaccactcttttcctttgtataatgttgttttattcttcagacagaagagaggagttatacagctctgcagacatcccattcctgtatggggactgtgtttgcctcttagaggttcccaggccactagaggagataaagggaaacagattgttataacttgatataatgatactataatagatgtaactacaaggagctccagaagcaagagagagggaggaacttggacttctctgcatctttagttggagtccaaaggcttttcaatgaaattctactgcccagggtacattgatgctgaaaccccat

SEQ ID NO: 13 - DNA, 900 bp, Artificial sequence
  synthesized
tcaaatctcctgttatattctagaacagggaattgatttgggagagcatcaggaaggtggatgatctgcccagtcacactgttagtaaattgtagagccaggacctgaactctaatatagtcatgtgttacttaatgacggggacatgttctgagaaatgcttacacaaacctaggtgttgtagcctactacacgcataggctacatggtatagcctattgctcctagactacaaacctgtacagcctgttactgtactgaatactgtgggcagttgtaacacaatggtaagtatttgtgtatctaaacatagaagttgcagtaaaaatatgctattttaatcttatgagaccactgtcatatatacagtccatcattgaccaaaacatcatatcagcattttttcttctaagattttgggagcaccaaagggatacactaacaggatatactctttataatgggtttggagaactgtctgcagctacttcttttaaaaaggtgatctacacagtagaaattagacaagtttggtaatgagatctgcaatccaaataaaataaattcattgctaacctttttcttttcttttcaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagct

SEQ ID NO: 14 - DNA, 900 bp, Artificial sequence
  synthesized
cttgacaagtctcctgctcctcactatgaagatcactgtcccccagccctgtgctccccgcactgtgctgcacgtccacctccattccactgcccctcccatccccccatcttgatagcacccttcccaggtgtcaagctgcccctcctagagtgtcctgcctaaaccccctctcctggctcctcccgctacagcatgttctctgaggacactaaccacgctggaccttgaactgggtacttgtggacacagctcttctccaggctgtatcccatgagcctcagcatcctggcacccggcccctgctggttcagggttggcccctgcccggctgcggaatgaaccacatcttgctctgctgacagacacaggcccggctccaggctcctttagcgcccagttgggtggatgcctggtggcagctgcggtccacccaggagccccgaggccttctctgaaggacattgcggacagccacggccaggccagagggagtgacagaggcagccccattctgcctgcccaggcccctgccaccctggggagaaagtacttctttttttttatttttagacagagtctcactgttgcccaggctggcgtgcagtggtgcgatctgggttcactgcaacctccgcctcttgggttcaagcgattcttctgcttcagcctcccgagtagctgggactacaggcacccaccatcatgtctggctaatttttcatttttagtagagacagggttttgccatgttggccaggctggtctcaaactcttgacctcaggtgatccacccacctcagcctcccaaagtgctgggattacaagcgtgagccactgcaccgggccacagagaaagtacttctccaccctgctctccgaccagacaccttgacaggg

SEQ ID NO: 15 - DNA, 900 bp, Artificial sequence
  synthesized
cacaccgggcactcagaagacactgatgggcaacccccagcctgctaattccccagattgcaacaggctgggcttcagtggcagctgcttttgtctatgggactcaatgcactgacattgttggccaaagccaaagctaggcctggccagatgcaccagcccttagcagggaaacagctaatgggacactaatggggcggtgagaggggaacagactggaagcacagcttcatttcctgtgtcttttttcactacattataaatgtctctttaatgtcacaggcaggtccagggtttgagttcataccctgttaccattttggggtacccactgctctggttatctaatatgtaacaagccaccccaaatcatagtggcttaaaacaacactcacatttattctgctcacatatctgtcatttgagcagggctcagcggggacagctccttctgtcctactctgtgtcaggtggggcagcttgagggttgggctggtgtcacctgaagactcattcttctgtacgtctgacaggcaatgctggctgttggctgggggcctcagtgccactacggaatagttggctaggacccctccatgtgggctagttgggcttcctcatagtatggtggctgggttggagggtgtcccaaaaagaaaggaggggatagagagagaccacttttcataacctagccttagaagtcacacagtattacttctgctacatatatatgttttaagaggcagggtctcactctgtcgcccagtctggaatgcagtggtatgatcacggctcactgcagcctcaacctcctgggctaagtgatcctcccacctcagcctcccgaatagctgggactacaggtgtgagtcaccaagcccagttaatctttagttt

SEQ ID NO: 16 - DNA, 23 bp, Artificial sequence
  synthesized
tataaggtggtcccagctcgggg

SEQ ID NO: 17 - DNA, 23 bp, Artificial sequence
  synthesized
agggccggttaatgtggctctgg

SEQ ID NO: 18 - DNA, 23 bp, Artificial sequence
  synthesized
ctcctgttatattctagaacagg

SEQ ID NO: 19 - DNA, 23 bp, Artificial sequence
  synthesized
tttcagcatcaatgtaccctggg

SEQ ID NO: 20 - DNA, 23 bp, Artificial sequence
  synthesized
ggcactcagaagacactgatggg

SEQ ID NO: 21 - DNA, 23 bp, Artificial sequence
  synthesized
aaggtgtctggtcggagagcagg

SEQ ID NO: 22 - DNA, 22 bp, Artificial sequence
  synthesized
ccatagctcagtctggtctatc

SEQ ID NO: 23 - DNA, 22 bp, Artificial sequence
  synthesized
ctcttcgtccagatcatcctga

SEQ ID NO: 24 - DNA, 20 bp, Artificial sequence
  synthesized
cacaccttgccgatgtcgag

SEQ ID NO: 25 - DNA, 24 bp, Artificial sequence
  synthesized
gcactgaacgaacatctcaagaag

SEQ ID NO: 26 - DNA, 22 bp, Artificial sequence
  synthesized
tgctccgggtttgtctcagatg